SEQUENCE LISTING (13 sequences)

SEQ ID NO: 1 - DNA, 5373 bp, Homo sapiens
gaattcctgcaaacccagcgcaactacggtcccccggtcagacccaggatggggccagaacggacaggggccgcgccgctgccgctgctgctggtgttagcgctcagtcaaggcattttaaattgttgtttggcctacaatgttggtctcccagaagcaaaaatattttccggtccttcaagtgaacagtttgggtatgcagtgcagcagtttataaatccaaaaggcaactggttactggttggttcaccctggagtggctttcctgagaaccgaatgggagatgtgtataaatgtcctgttgacctatccactgccacatgtgaaaaactaaatttgcaaacttcaacaagcattccaaatgttactgagatgaaaaccaacatgagcctcggcttgatcctcaccaggaacatgggaactggaggttttctcacatgtggtcctctgtgggcacagcaatgtgggaatcagtattacacaacgggtgtgtgttctgacatcagtcctgattttcagctctcagccagcttctcacctgcaactcagccctgcccttccctcatagatgttgtggttgtgtgtgatgaatcaaatagtatttatccttgggatgcagtaaagaattttttggaaaaatttgtacaaggccttgatataggccccacaaagacacaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaagaaaatatgcctattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagttcttgggtacttaaacagaaacgcccttgatactaaaaatttaataaaagaaataaaagcgatcgctagtattccaacagaaagatactttttcaatgtgtctgatgaagcagctctactagaaaaggctgggacattaggagaacaaattttcagcattgaaggtactgttcaaggaggagacaactttcagatggaaatgtcacaagtgggattcagtgcagattactcttctcaaaatgatattctgatgctgggtgcagtgggagcttttggctggagtgggaccattgtccagaagacatctcatggccatttgatctttcctaaacaagcctttgaccaaattctgcaggacagaaatcacagttcatatttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccagattggctcctattttggtagtgtgctgtgttcagttgatgtggataaagacaccattacagacgtgctcttggtaggtgcaccaatgtacatgagtgacctaaagaaagaggaaggaagagtctacctgtttactatcaaaaagggcattttgggtcagcaccaatttcttgaaggccccgagggcattgaaaacactcgatttggttcagcaattgcagctctttcagacatcaacatggatggctttaatgatgtgattgttggttcaccactagaaaatcagaattctggagctgtatacatttacaatggtcatcagggcactatccgcacaaagtattcccagaaaatcttgggatccgatggagcctttaggagccatctccagtactttgggaggtccttggatggctatggagatttaaatggggattccatcaccgatgtgtctattggtgcctttggacaagtggttcaactctggtcacaaagtattgctgatgtagctatagaagcttcattcacaccagaaaaaatcactttggtcaacaagaatgctcagataattctcaaactctgcttcagtgcaaagttcagacctactaagcaaaacaatcaagtggccattgtatataacatcacacttgatgcagatggattttcatccagagtaacctccagggggttatttaaagaaaacaatgaaaggtgcctgcagaagaatatggtagtaaatcaagcacagagttgccccgagcacatcatttatatacaggagccctctgatgttgtcaactctttggatttgcgtgtggacatcagtctggaaaaccctggcactagccctgcccttgaagcctattctgagactgccaaggtcttcagtattcctttccacaaagactgtggtgaggatggactttgcatttctgatctagtcctagatgtccgacaaataccagctgctcaagaacaaccctttattgtcagcaaccaaaacaaaaggttaacattttcagtaacactgaaaaataaaagggaaagtgcatacaacactggaattgttgttgatttttcagaaaacttgttttttgcatcattctccctaccggttgatgggacagaagtaacatgccaggtggctgcatctcagaagtctgttgcctgcgatgtaggctaccctgctttaaagagagaacaacaggtgacttttactattaactttgacttcaatcttcaaaaccttcagaatcaggcgtctctcagtttccaagccttaagtgaaagccaagaagaaaacaaggctgataatttggtcaacctcaaaattcctctcctgtatgatgctgaaattcacttaacaagatctaccaacataaatttttatgaaatctcttcggatgggaatgttccttcaatcgtgcacagttttgaagatgttggtccaaaattcatcttctccctgaaggtaacaacaggaagtgttccagtaagcatggcaactgtaatcatccacatccctcagtataccaaagaaaagaacccactgatgtacctaactggggtgcaaacagacaaggctggtgacatcagttgtaatgcagatatcaatccactgaaaataggacaaacatcttcttctgtatctttcaaaagtgaaaatttcaggcacaccaaagaattgaactgcagaactgcttcctgtagtaatgttacctgctggttgaaagacgttcacatgaaaggagaatactttgttaatgtgactaccagaatttggaacgggactttcgcatcatcaacgttccagacagtacagctaacggcagctgcagaaatcaacacctataaccctgagatatatgtgattgaagataacactgttacgattcccctgatgataatgaaacctgatgagaaagccgaagtaccaacaggagttataataggaagtataattgctggaatccttttgctgttagctctggttgcaattttatggaagctcggcttcttcaaaagaaaatatgaaaagatgaccaaaaatccagatgagattgatgagaccacagagctcagtagctgaaccagcagacctacctgcagtgggaaccggcagcatcccagccagggtttgctgtttgcgtgcatggatttctttttaaatcccatattttttttatcatgtcgtaggtaaactaacctggtattttaagagaaaactgcaggtcagtttggatgaagaaattgtggggggtgggggaggtgcggggggcaggtagggaaataatagggaaaatacctattttatatgatgggggaaaaaaagtaatctttaaactggctggcccagagtttacattctaatttgcattgtgtcagaaacatgaaatgcttccaagcatgacaacttttaaagaaaaatatgatactctcagattttaagggggaaaactgttctctttaaaatatttgtctttaaacagcaactacagaagtggaagtgcttgatatgtaagtacttccacttgtgtatattttaatgaatattgatgttaacaagaggggaaaacaaaacacaggttttttcaatttatgctgctcatccaaagttgccacagatgatacttccaagtgataattttatttataaactaggtaaaatttgttgttggttccttttataccacggctgccccttccacaccccatcttgctctaatgatcaaaacatgcttgaataactgagcttagagtatacctcctatatgtccatttaagttaggagagggggcgatatagagactaaggcacaaaattttgtttaaaactcagaatataacatttatgtaaaatcccatctgctagaagcccatcctgtgccagaggaaggaaaaggaggaaatttcctttctcttttaggaggcacaacagttctcttctaggatttgtttggctgactggcagtaacctagtgaatttttgaaagatgagtaatttctttggcaaccttcctcctcccttactgaaccactctcccacctcctggtggtaccattattatagaagccctctacagcctgactttctctccagcggtccaaagttatcccctcctttacccctcatccaaagttcccactccttcaggacagctgctgtgcattagatattaggggggaaagtcatctgtttaatttacacacttgcatgaattactgtatataaactccttaacttcagggagctattttcatttagtgctaaacaagtaagaaaaataagctagagtgaatttctaaatgttggaatgttatgggatgtaaacaatgtaaagtaaaacactctcaggatttcaccagaagttacagatgaggcactggaaaccaccaccaaattagcaggtgcaccttctgtggctgtcttgtttctgaagtactttttcttccacaagagtgaatttgacctaggcaagtttgttcaaaaggtagatcctgagatgatttggtcagattgggataaggcccagcaatctgcattttaacaagcaccccagtcactaggatgcagatggaccacactttgagaaacaccacccatttctactttttgcaccttattttctctgttcctgagcccccacattctctaggagaaacttagattaaaattcacagacactacatatctaaagctttgacaagtccttgacctctataaacttcagagtcctcattataaaatgggaagactgagctggagttcagcagtgatgctttttagttttaaaagtctatgatctgatctggacttcctataatacaaatacacaatcctccaagaatttgacttggaaaaggaattc

SEQ ID NO: 2 - DNA, 30511 bp, Homo sapiens
atctgaattgtaaatacatattaagagaaaaagtatagagaattgagattgtccagatttatcaaaagaggcaggatataaagggaaagatttaagtagctgaaaagagatttagtatcaatggaatgaagggatggtgataaagaaggtcatgctttgtgaggaaaaatgagcagatcatttatgaatagaaattctggacaataaagttgagagatgagccaaacctagactcagagtgggtgacattgcgcaagaaagacatttagccttcatgagtttcagtttccttatttgtaatttgttaataatagcttggttttgctgttccacaggattgttgtggcactcaaatgagtcaggatgattctgacataggcagacataagcattaagaaacagtaagatgcgagttggaataaacattataatttaaatagaggaaaaatactggaaggaaatacatctgaattaactgaacttacagtagaaagaaaagattatgaatgatttttcccttgagttttctctatattccaaatcattattgaagaacatatagtaattaaaatgcattttattaaaaaaaaatcctaggtgctgagaaagtaagagtgaataaaagaaacaaggtttgacttctcaaggagcttacattttagtaggcacagacaaaagaatatggctatataagataattttggataattgctctagtagtgatacagtgatgaagaagggtgctgctgctacacagagggccaagggagagcagagacctatttgagcaaagtcctacatgccgtattaggtgaggatgttataggcagtggaacaggcagctgcaaagcgcataagcagaatgagctgcatgtgttcaaaaaacagaaggaaagtgagtgtggctggaacacagggagggaggacgagagtggtcggaagtgaagtcagaaagatacaccagggctcaatcatgaaggacctttgaatttacagtgaggacattggattttattctcagttcaatggcaagacataagttaataatatatatctatgcatatatagtaacacatatatgcctatatatgtgaattgctgtataattgaactgctaaatgataaaatgaactccatcataatttcctgccattaaatgatctgcccaggtctaattaatgggggaaaatgaggttgttttatgtacagtcacgtgtcctttccagaattaggtattttaaaggtgaaagcagtttggcatcatctctccacgctaatatgttagatgattgataacatattcaaatagcatttattgagctctccacaatggggtgaagataaatgttggttgaaaacaccaggtattttgcatgccaattctggatctctgcttaatttgctgttattttcttttgcatttttttattattcaaaaataaaaaggagcaatggagcatatgaaaaatagatgttaaggagaaaatttgcagatgttaagctttgcagattttgctatagagtatttcagaatgagtatttgtgtttctcttcatggcagcacattatttgtaagaaataaaaagctgaattccttgtgtggacaaatccctgatacagtgattttggcacttacagagggacgttaactacttaaagtccccgttgcttcatcattgacttcattatcattttggagcactctacagaatctgctccctgaaatgcagaggcgcaatggaagagcaagcctttttcttccggcctttcagtgcctaaaacaatagacatatgaaataccatgcacttgcaatgctgcctagaaagtaggacatttctttgtgaaagcacatgttaatggaactttgatgtcaattttaaagagtccattctgtcttcagtgtctgcgaaggtaaatattgatcttaagcaacatgaggcatattcccatctccagagagagcattaatatccacacaaccttctttctgtaggatgaagaactggcagcattaatgctgtcagtgtcatacagagaaacatgatttgccttattcctttccttcattccctttccatcagaaataagaattttcaaaaaaaataaaataaaatctatcaaggctttatatgccttggtctttgttctgttttttgaaatgattagcatttctcaatcattcagaaagaagcatcaaaaacaaaacacttttgacagttcccactggaataaaaaagatatgacaccaatccctacattttcatcctcaaaaatgataatgatcacttctgaaagaaaatgataacaattttcaaggagctgtgattttctatctcctttgatgtagtcatcaatgccttttcgacagatgtttcatatagagtccatgtcaagatgttcaaggtgattcagaataaatagatgatacccttcagcccttgcccttgggtggcttagagtcttgttgttttgggaaggacaaatataactcaaattacttaagaatacttagagataatttagttgttttaagtcttgtgttcaaattttgtcttcatcctttcctctttccagctgtgagatatacttgtcccctggaacctcaaaattctctgcaaaacaaagataatattagtaactatcttctagcatttccatgaagattagtaagataatgaattatgcatatatagtatttagcttggtgccgggcctccaaataagtacttaaaaagcagtagttattttttctgggtaaacgttgataagtaataattattaggtcaagcaagttttcttaaaaatggcctttgagaattatacgacacacttatattcatgaattgtaaatatgtgctatttcatatttttcttaataggcattttaaattgttgtttggcctacaatgttggtctcccagaagcaaaaatattttccggtccttcaagtgaacagtttggctatgcagtgcagcagtttataaatccaaaaggcaactggtaagaatattctcttctttatgatttcagtaaaaatacaaaataaatttcatatttgacttcaattgctttttcaaattggtgcctgactgtactataaatgggtggccctcaaatatccttaaatgaacaatctttctttgagagttgacaactgcagactgatcattaatgggatggtataatggttcatttttaaaaacctggatagaaagttttctctaaatattcaaaagcaaatttataataccagttttgaccattaatacataggtaggtagattgatagaatgatagatttgtcttcatttctgatcatgtaaactgatattcattgaactttgtaaatcattacctagttattaatgctatctctggaccatttctaaatggtagaattgtttcctacatttttttctttagcaaggttttgtctacttgcttcattctaagttaggcttttgccctccttgtggtgaagggtcctcacaaatatcaatgcaaaatgtacaaaggtggctttctctagcatattaacaccttttgtttttgccccaaagtgctcccagtttttacatgctatgtgatggttacattttcaaaccatggcttttctacccaggtgaaaaacaaccacctcaatggagatggaaatctagttcccataatactaatgcaagtgaacccagagaaatgaggtaatttgaaagttgtatttgggaagaagcgctgtagctcctattctgccagagttcatgcccactggtggacacaggtcctgccatggggaccttatctccttgttgcagcaggaggctccaaccatccccactcaccctggaacttcagattctgaatctccactctctagagctcttttcagtgattaaatgggctgttgttggcttatcaaataagaaccgtaatgtatggccaagtgcagtggctcacgcttgtaatcccagcactttgggaggctgaggtgggaggatcacttgagcctaagagttagagaccacctgggcaacacagtgagacctcatctctattaaaaacctgaaaatcagctggatatggaggtgcatgcctgcagtcccagctactcaggaagccgagctgggaggttggcttgagcccaggagtttgaggctgcagtgacctatgttggcaccgctgcactccagcctgagtgactgagtaagatcctgtctcaaaaaaaaaagaggaaaaaaccgtaactgtaattcataagcctttttttctgtgtgaacttccatttgatactctaaacaattgaattcaaagaaaggttcaggatcacatgttggggcatgcctgtctatttgatgaggaaacaaaatgaaatcatcccctgagagggcagcatttctcagcaggcatcttggaggatgtaaatcgtgagctgggttttaagtagtgaggatgaagaggctggagatgagtgttaactaatgtgaggagacttagaaaacaatgcatgtagatacagtttgtcaattgtctagaaggataagaacatctggttggaaaccaaagattggcaattattgtaaccatttgggatacgagagagagttgcatgtctaacaattatcaaatgatgctttgataatcataaagtggtgaggaggctagaaagacaatatggtcggtatattttgaccttggaatctagcagatgaagtagctaatgacccacagttttaaatattcagccctaattttttccctcagactgctatattaaacaattcattcaacatctccactttaatgcccagtagacataccaaactgaactgggagaacacaagtatctgattttctgcacaagctaatgaggccaaaaatctttgacttgtcttctttctcctcccctatatcttatccatgagccagtcctgttaatcctatcaccaaaataccaattatacctggccgttcctcactgtctctcctgatgccaccctgatggaagccaacaccagctcttacaggcactgctgcagcagcttctccacctgccttcagcttcccttcctgccagctttattctgctgtctttgctctagccagtgtgaactgttaaaaacatggctcggccaggaacggtggctcatacctgtaattccagcactttgggaggccaaggcgggtggatcacttgaggccaggatttcaagaccagcctggtcaacatggcgaaaccccatctctactaaaaacacaaaaatcagccaggtgtggtggcgggcgcctgtaatcccagctactccagagtctgaggcaggagaatcgcttgaacctgggaggcagaggttgcagtgagccaagatcaagccactgcactctagcttgggtgacagagtgagactccatctcaaaaaacaaacaaaaaacacagctcattccctagattaaaattccttaaaggatgctctgcgattgggtgtcagcctctctcctattatcatcttttacaccttctcctctgctttctatgctccaagaactaagtcttttgtctgttccttgaacatgcctagctcattcctgttgcaaagcttttgcaaattattttctcctgcctggaagagtcttccctaagatcatcactcaaatggttttcatctttcagtctcagctcaaatgtttcttcctcatagacctttcagagcaaccctatctatatagcactcgactgctctctgagtcactccagccttacaggtggatttattttctaatgattagtactatcttaaatgatctatacttgtttatatgctcattatctgtctcctgccactgtctctgagttcagggacactgttttgttgactgctgcattctccacacccagagtgatatctggcactgagtaaaggatcaacaattatttgttaaatgactaaatggaagtcccaattctaaatgattcatcccaagtgctatacaaagtgagctatagtagactgacttaagatgtttgagctgaaagggagcttcagatgaactccactgttctagttcatagacatggcctagctgaggacatctggcaccgagtttgaagcaattcttttaagtaacagctttcctggacagagtaaatctaagagccaccatcagtaatagacattattgaaggtgataagcctttcaaggattgtaaggggctcaaccactggccatgctgctggttggaagttgaacaagacgaaaagggacacagagttgagaggatcccaccaggcagagttctgggaacatgagaggcagccatatctcctttctctgtccaggattacctctaagtatccaagacattgttgaaagacaagtatatttgtagtttatagtgagatcctagtgctggtaagaccagggacactggacatttctaaatcctgcctggaccttctcatgacatccctgaatgatgtataaacacaggacaataggtctgggaagcaaggcaccactctattattggctgggccagggctttgaactccagtatagagctttctggcaaggtgtagcactgagattaggctcaaggcctaaccaccagatgatacaagcataccccaccatgcaagaggaagagacagcacctgtctgacctgaagggctgaaccatatggaattcagaagcaggaaggacagagaacaaaagttggccaagcaagtctgtctgggtccttttgttgatctggtcttatgaagcaaagaaacaagagaggcaaaaccatgggcatgattttctaaactacatgaggtgcaaaaaatgcaaaacatagttgttggccttaaggagtgtaaaatagagtcagtgaggaagatttatatgcagaataggcaactttgaataatatagaagacatcagtcagtgtaaaccccttgcagttaatggtgtttgttttctttcagtgtcactagctataataacttaagagctcattttcttttcccaaggcctgggtcattagagagacaaggttttctcatgacctctgtcatttgcccagccctgccaaggtaccttgccacagagggcagggctgtggccaggatccaggtgaatgggttatcccattacagtgctagagtgggccatgttaggacaagcagtcttcacgactctcaggctcaaggaaaattatatgaaacttggaacccagaattgcctgtttgttttgacagtgggccctgtgcccttctctactcttaatttatgacagttgcataggcattatggaagtagcagatgtgggaaggactgtttaaacattgactttccttctgatactaatcaagttagttttggacacagaaattggtatgaccatgaattattcctttagcaaattgcgagttctttttccatggatttatcatgtaatacatgcacacacacacaactttgttttctttattcccttactgcttattctgaagctgtcattttggtatctttttcctctgcttactttgactttctttgtaatctttctatctaagtcctgacttcactcctttccctcagacaactcactgaaggctctagtctgagtattcttaaatcacactctgttatttttaaagtacaattttagctctgtcatgtgtgccatgttttctaagaatttatgggcatatttacatcaggatctaccagattttaacaaaataatcttaactcttttgctagtttcatttatgagactttcattgcactatgaacttcaactctagaaacagtggtgacatggctttgataattctggagcctttttaacagtctcaagtgagcgggataattttaaaacaagctgacctcccaagtagagtacctgggcagccctctggctgtaagtaaggctgtggtctgttctcttttcggtgtaggcggagggccatcccgcttcagagccctcacttcacacacaggatgttccaatgtggatgatgcaattcttatcacaaacaatctacaaaggattgagatatgtttaaaatcatttcctggagtttgtggtgatatgcactctttctgtttgtctaatctgaaggcctgttgcaagaacaatctgagctcattatgtgactcaaaaaccagagagaactgcaatctctctgtgtgatgaggactgactgggaattagggtatttccagttgtagaaatgtctttcattctaacaaggactagtgcattctttttccttaaagagagaggagaaatagaaagaacaagtgtcatagcaaaaaataaaaataaaaaaaatccacctatatatgagcttatagttttaactttttaattgagaaaaactttaataaagctctacatgtgttatgaatatagcattgtcatccacagatgtgcctataggagaactttaaataaaaaaaaaagtttaattctataacagacatcaaagattatgtcacgtgatactttgataataacaccacctggtatttattgagtactctttccttgccaaacatgtaatacccataattactctaggagctttgcaaaattattaccccctttacaaatgagaggagtgtgactcagaaatgttaagtaattaacctaagattacagagctgataattaataaagataggggaagctaatgcaaaatgagacaaaatgttccaaacgtacatatttttgtaagtatccaagaaggagagtgacaggatgtagttggcatttataaataatatttaaattttattttaacataaactttatgaagtaagagaaactttcagaatcttgaatgaaaaattctgttagagaatttccaatgttagcatcattggctttaccatcagctgtttcctgggcaaagatgatagttaccatccatcttttcatcctatcatagacaggaatttcttgtatgaatatactcaggagccggagcacaagggttttaccctcagcttccccaccactccctgggtgactttgggaaagtcacacaacttctctggagcagaaagtatgccatccttatcaaactggaatgttatgagcaccttgtgtgagaataaaagtaaagcacttcagggctgggctcggtggctcatgcctgtaattccagcactttgggaggccaaggcgggcggatcacttgaggtcaggagttcgagaccagtctggctaacatggtgaaaccccatctctaccaaaaccataaaaaattagcagggcgtggtggcaggcacctgtaatcccagctccttgggaggctgaggcaggagaatcacttgaacctgggaggcagatgttgcagtgagctgagatcataccactgcagtctagccttggcaacagagggagattccatctcaaaaaaaaaactgggtcctccctacctcttcctatcccctcaaccaattctcatctgtcatggatgagcttccatgcatattggattagcttcatgaagtccatttcttcttcttggccaagaatgctattctattccaatcctctttccaaaataatcatgtctttgtcttttatgatatagccttttaaatattagccaattatcatcctttatatctctttcaaagataaaagtctcttaccattaggttgtcctggcagtgccatttaacatgccaacttcatttattcacagagtttgcttatatcttatttaattaaattaattaattaattaattaatttgagacagagtctcagtctgtcaccaaggctggaatgcagtggctcaattttggctcactgcaacctctgcctcctgggttcaagtgattctcatgcctcagcctcctgaatagctggaattataggcatgtgccaccatactaggctaatttttttttttttttttgagacggagtctcactcttttgcccaggatggagtgcagtagggcaatctcggctcactgcaagctccgcctcctgggttcatgccattctcctccctcagcctcccaagtagctgaaactacaagcgcccaccaccatgcctggctaattttttatatttttagtagacacgaggtttcaccgtgttggccaggctgatctcgaactcctggcctcaagtgatcccatctcagcctcccaaagtgctgggattacaggcgtgagccaccgagcctggccctccttttattttaaattcataacataaatattccttttcttcttccattcaattttattttttactcataacttagtccagtgtttgattctataaagacattagaaatccaactgtaatatattcttacacttttattttaactttaaatacttactgttaaaaattttataaatacttattatgctattgctgtacaattaagccatcctcagcactattaatacaggtatccaaagtggcatgtctcatctcaacaatgacaaccccccaagaggcggaggttgcagtgagccaagatcacgtcactgcactccagccggggcaacagtgagactccaactcaaaaaaaaaaaaaatctatagtaggaaggacactttaaatatctcctgatttgcttcttcgttccttcactctggatacaagtttgtgtgtgcacctacaaacacatgaatacatacacacccattgtttgtgacttgcacgtgggagagatatgttgctccagaatgtctgctgtgctttaatgatctataaaaatcctccatgcctcaccaacaagtcttataattttaagctttcctctgtggtaatgtgtgaatatctgtttctggaataaagttcatttacaaaggtgactgggacttacacacacaccttcctagtatctagttgtctaagacatttacatccatctaactccatctaaagaaggatctggggaatgctaagtggcacaagtggcaactatttattaacttgctgtgccccatatgatgggaaagcaaacagtgcttctctagaagagttctaagggaacttaatatggtaaaaagaaccagcaattttgagcaaaagggcaggggtgcagatatataggttttgctttcacttagacccttgaagtcagtttctttaaatgaaaacataaatagttatacacttgacctcacagagtgtttataaggatcaaaggagataatgcacataaaaaatgctttatgagctataaagcagtttacaaatgttgctattccagtttttatcattgatgaaacaaccatctgccaaagcacctagttgaaaggtactagctcatgagctttgatttgcaaaatgtgatggtcttaaaattatccccctctctccctccaatagccaagaacaggaatttgtatcatcacaaggctttagggttagtattttagcttttgattttaaattcaagaaaactgcatgtcggatgagaggaagtaacataattagtttttattgattatatttcttctagtgccaaagccaatttctggaatttttatgagacagaacagtaagcttaagatgttttagcaatagtgaagtcagtggactctttatcgtgaaacggaaagatgctgaggatctagccatcccgctaactgaggagacaatgtggaagcctaagaagtctgaggcagcataatgaggcctaaatactaaaattgcctttcaatatatgccaaagaatttaactattaaatttatgatagcagtatttccaaataaaacctaaaaaatgaaatgactctttttacctgtcttccttcttcccagagacgggcagccttcacgggtgaattctttccatcacaaattcattttatgtttaccagcagaaagcaggtaccatttagagctcaccaagcaccttgtgttgtccgtggcctggaactaaacaatataagaaacaatatttatatttatatactatagacttggaaaatttttacgtgcatgtagatacaataagtttctaatccaaagtaaaatatccccttggatggctcatagacctctcaaacttaaaatatctcacagtgaactcttgagctccccctatcgcaaaatcttgctctactgacagcatcccctttaatctgatggtagtgactagtttcttttagcttctcagccaaacacctttaggctgtctttgactcgtattcattcattcattcattcattcattcattcattgaaagaccaactcctcaagaagtcctgctgcccctaccatcacactctatctaaaacaagaccacctcttaccactttcccttctgccagccctgtatgagcactccatcatctgttgcctggatgattgcagttgtcttctaagtactttacatgactcacctgtgcttcaatacaatgattcccaacagtgtggccttcaaaattcttctacaatatgtcagatcacatcatgctgtactcaaaaccctccaatggcattctcatttccctcagctggaaagctgaggccttctattaatcattacgtacaagtcactacatgatctatcattaccagtaggactaccatctccttccactatccccctcactcattctgttccagccacagggcttttgcatttgctgttcccgcagcctagactattctgccactagagtcttaaggtttactctttaaccttcttcaagtttttacttaaatattaccttctcagtgaggccttccctgaccattccgatgaagattatactgctctccaccccacactcctaactccagcctgcttatttttctcccaaagcactgatcactaccactatatggtttacttacttattacatttactgtctattttctcccactagaatgtaagctcctcaggctagaaacttttttactgatttattcactagtatatcctcagtacctagaatgaggtctggtacataataggtctggccaccacgtatagactcacagattgtatactgcacaaggcctggacacaccattcaaggaatggtgtcccctggggttgtgtaccatttcagtccccaagagcccaataaacatttgttataaaattgaataaaacaaaatgttttgccctactattttaagtacatctctgtcagtaatctctggtaaaatctctagtattgaagaaaacacttggagaatttacagactcacagaccaaaggaagatatttataacagcccctagattgtgggtttaaaactgtcctacctacaccaccataaatactgcttgctaactaaacaaagtattttgaattccactggaggtaggcaaattggctgtgttttgaaagtctacataatcactattttcccacaagataattgaacagtaagtgttatttaaagtagtcttggaagaattcccagtttcactggaggaaaagaaaagtttatatcttgggattttgttttgttttgagaatcagcacacatgcctgttcatgcaactcttttgtctggttgggattaaattaaatccattacttcaaagaggtttcgccatacttcaacaggtatactcaccaacatatccaatttcccaccttggtaacagtttctcttggtgtgtctcttcctgcacgtaaaatttcatagagaaatataacagcctccctcatcttcctgtctgattaatttaagaatgccacaattttcttggaatagcctcttggtgatgtcctagcctcttcttatgagggtgagttgtcaggaactaggatttcaccgaggccttagggtgtgaatgaggaggagagtctgctggaaatgtttctaataaagaaggtaagcaaaggtcagtgacttctctgtagtcaccaacatgacctgtagcctacttattaatgtggttcacaatatttaagaaatgagtcatatatgatgaggaaacccttgttcttcaagagttaccaaagtggtctggttttccactattttctattgccttgtgttaagagtttaagatagcataatactgtcagagatgtatttgcatgtatcttttagggaatttcaagtgcttccacatccttcttgttaagtgccacacaactgcacagatgcttacttttctcttctttctgattaaattagaagagctgcagttttcacagaggtggtcatgatgttatgacccctcctgtgctgcaaggttatcaaatcctgaattcttagaaaaggtggtaggaggtgacagggagagtgtccagggagtgcttagttgagagaaaaaaaccaaacagtcttctcaggaagaaaatagctagttgttgcagctttgctaccagaggctctgataaaaaaaaaaaaaaacagttaaattttagatcagacacacttctctgttgtttctcagcaagcctctctcgggctgtggtgaggatggttggaaatgtgcataatagaatcacctgaatttggtatttgcatttggtattgtctgaacataccctgttcctacctgtcattgggtttgtatttggtgttgtctgaacatgccttgttcctacctgccattgagaacagtgtagcctaggccccacagtatggatgtaccctcccaggacagctgtgcttcgtgtccagtctgcagccccttcatatatcacacagccacacaccccttcacataagtgtggcctgactccctgtgtctggtttctaatccatgctagttctagaacccacctcatctgctaagatcattttcccaatctgggccctctgccaggacttcctgcagctaagaggaaggacactagcctaagcgttaagaacctgggcttccgcacttggttttcactagccatgggaacatgagcaagtcactcatcaatctgtgtctcagtgattccatctgtaaaatgaagtgtaaaatggggataagatcatccccggtggccaggcacagtggctcacaccagtaatcccagtgttttaggaggccaaggagggtggatcacttgaagtcaggagttccagaccagcctggccaacatgatgaaatgccatctctattaaaaaagtgcaaaagatagctgggcatagtggtacatgtctataatcccagctattcaggaggctgaggcaggagaattacttgaacccaggaagtggaggttacagtgagccaagatcatgccactgcactccagcctgagtgacaaaagacgagaccatctcaaaaataataataataatttctggcttcttctgcagatgaaattagataatctttggaaaggcttacacatagtaccagatgttgtaaaatttactataattaataggaattaattaatgaatgccaaggggcagagccacacttcctatgatagttccttgctataaggtgctattttgttctctacatttactccatagtaagcttttgtttgagaaaaaaaatgccagtttggtgcgtagtagatacgcagaggctgagaaaggaacagatgacacaataacacaatggtaacagaatgtataatgcttccctctcaacttggttgatttgttttttttacatttacttggaccaaacaagcaaaaatacatgctccccagaagaacagaaataaggattttctttgggaggtggaggcgggcggatcacctgacgttaggaggttgagaccagcctggcaaacatggcaaaatcccctactaaaaatacaaaaattagccaggtgtggtggcgcgggcctgtagtcttagctacttgggaggctgaggcaggagagtcgcttaaacccgggaggtggaggttgcagtgaaccaagatggtgccactgcactctagcctaggcaacagagtgagactccatctcaaaaaaaaaaaaaaaaaaaaaaaaagtaaggattttctgcttaacttttccttaatcaggaaactcttttttaaatgatagcatagaatcatccattcatttgcttgttcatttgctgttgttttttttaactcatttgttcaacaagtggttattcattgctacaggcacagaattcctagccctcatgtacctttcctctctagtgagggatttagtcagtaggaaggcagctatggggcaatatgattttttttaatgggatgtggaaaggtcagaggctatgtaggtacttagggttacctcgagttaaaagagccgggggacttcctagaggaagtggtgtcaaacaggagatcaaactaatgagtagacattgccaggcaagaaacagcatgtatgaaggtttagaagcaacagagactctaatttgaaggagaagctaaattcgatatgactgaaatgtgatttcaggaagacacatagaaaaagccagggttagagaagaaagagtcagattctttcctttttacactttaagatatgtccagcatgcaaaaatcagagttaatgctgtgtaatactactatgaaatgaagacctgtacagatggagaaaatcaaggctctggaagacaattttcttggtaccgactctataagattcctaatttcttctgagctatcatgaatttgaaagacatcagatatgctcaaacacgcgttccaattctgcactggttccctcccttcattaaagtggctattgggacttactaacatccaacattcagatttctatctttttcaaaagccttaacctatctgcctggtattattgtaaacctaactatggtaatattttaaaaggtaaattatggtaaattattagctcataagaagaaaaaatctttcaactctaacgttaaggctgactgaattaaagtgtgctagagtgttctatcttagtttgagaataattttataagatgaacgaggagagaacttattaatgaactattaactttcaaaataatctgcattttcctttttccctaagtgttcatgctattctgtctgctaatacttcaggttaaaaccttcatcctttggtgtccatttttgtaaccttggaccattttaatgttttttctactggtaaactagatcatatattccaggtcatttggatttttattttacttgtcacatatatctagttttactagatttagaagacagcagtctgctttaatccttttgtatctaccgtacctcaacatggggcatatgttgtttaaatgataattataaaatatatatacagaaggctcatgaaataattttagtttctttgatattttaacccttcccaatactgacatgaaaaaaaatggaagcaaatatgcttccgtattaattgaatgctgctgagtaacctgctgcagtttctaaagatcaaactgatacctgggctctttctgtgcaatcttctgtggttctaagtgtcaggtgagtgcttctgcaagaaaacccatattctctaggtgaagttctgaaggaaatgaaactaaccgaaggaaggatgtaaacaccaatctcatcctgtattctagatatatgcactttgttacacataaagatagcttgtaatgtatttgttctgtatttttgctaagtatcctctcataaagacagttagcataaagccactaagataagatatctgctctctaaatgagtcaacatatgaaaatgcttagggcatagaaagcactcagcaaaggctagttgttgttcttggtttaaatttggtttcattctttagagctgtgctgaacaatacgtagccacgagccacatgtgactactgagcacttgaaatgctacttttccatattgagatgtgctctaagtgtaaaatctacactgaagttcaaagacttaggaaaaatgaatgtcaaatatctcattactaattttgatattgcatgctagtttggatatattgagttaaataaaatatatttttaaaattaatttcacctgttgctttttatgttcttaatgtagctggctactagaaaatttgaaattatatatgtggctaatattcgtagcttgcattatatttctattggacagcattgctttagagagactttctagggtgtgtggcaatgcagcagtgttacagacattgatcaaagctctttatcagctaagcagaaaggcagcaggtcaaatcatagctgaacaaatataaactgttcacattgaaatttaaatgtgtttttcactgtttgtgatcaggtttatctttaagaaactatacttaacactttgtgtctaataaaaaaaatgtgtttctaggttactggttggttcaccctggagtggctttcctgagaaccgaatgggagatgtgtataaatgtcctgttgacctatccactgccacatgtgaaaaactaaatttgcaaagtaagtttaaatttacttgcaaggcatgtgatttgtcttagactcaactgaaaaataacatcagaacaatcattgttctgtcttaaagaaatacagacagcttttttttgcccttatgtctttagtaatatggggcacaatcatatccaaatttccaaaacagtttggagacttgtcttaacaagatgcgtggaaaatgtgctatctcctggaccatttacctatagaccatggactggtttccatcaacatttagaataattttgatgattacttgcagaatcaaggctatccttgaatccgaggctatccatatttctttctctcctctttgcaaaataaaccattaagtttggaaacatcagtacaagggctgagtttggaggcaccaaaaacactctgtgtttcttgttatgtctaaatgatggcattctaataaaacaaatccacaatttggaattatttgctgttttaattatccccatctcagatgcctcctattaatgccaaaaatttactaaaggttcaagggtagacagctaaatgagtgacatttccatgtaatatgctgatacaagtaaaaagtcactcaatttgatcatcattacatcatcagcagcagcagcctaaaaaaacagccctgaggaatcacggagttcctctgaagaattgaggcatgaattagaaaagagtgttgtgaaaatcaagggaaagaagagtttcaaaaatagtgattaagacttggaaacaagggaagtggtgtttcttgcatatctctgtagacctaatatctagaataatttatggcacataatagggctcaataaacatattttcagtgaagtagaaggaatatcagtagtgtcaaatgctacagaagttgaaggggatgaggactgagtaaatgtcttcagcttatttaagcaggttgtgggtgatctagatcagtgatctccaaattttttgaacactcagtatatatatatatatatttctaataacacaaatatatacatttggagtgagtgttcaaagctttattatatagtccaaaaattttagtatatcacccctaacatttcatatcacccttaacttctgaacactcattcttaatgtatatatttttattatttttaaagctatatacatgtactggcctatcttctcaataataccttcacataaaaataaattttaaagaataacaacatgcccatagaagttctattattttctgtcttcactctaacagatcatattttgtgtaacttactcacctagaggataaatccaccagtgttatggggagaaaatagataacaaagaatttaggagtgtgtcagtagtgaatgtacaaggagtgaaagttgacctccatttccagaagttcagcagtgaagggggagctgggaaaaacaattctatgggatagcagtgtgagaagatttttctagtaaacaagagatgatactttttgtagggaacatggaggcagcagtaaaaagggatgatttgtccaggatagagttaactgattaaaattaatgtcatttgtacctgagaaatagagtcttaaaaagagttcttccttctctaagacacaaaggaaggagagaaaaattaggagttctaagaatgatctttacacaatgattagagaaactgagattaccctggaagtctgattcatcttctctgcagtagtcgttattcactgagtgatgggatctgagttgaggttggaggcttgaaaagaaggggtgaacttggctgggcatggtgtctcacgcctgtaatcctagcactttgggaggccgaggtgggtagatagcctgagctcaggagttcaagaccaacctgggcaacatggtgaaaccccatctctactaaaatacaaaaaattatccaggtgtggtggtgtgcacctgtattcccagctacttgggaggctgaggcaggagaattgctagaacccgggaggcagaggttgcagtgagccaagatcgctccactgcactccagcctggatgacagagtgagactctgtctcaaaaaaaaaaaaaaaaaaaaaaaaaaaaaaaagaaaggtgaacttttacaacagtcaccctgggaaataagggaaggaatccaattaagaataagtgacagaggcaatgatagcttggcagagatgagaaacatgactgaatgatggtattactaaagagaattgcaatgttcgataactcaagaatggatacagtggttgatgccctaggtcagtggaagacaggaggtggagagatttatttagtatattatatgactcgtctaatttccttttaacattcacccactattgctttgagccagattctatcatagggcattctttgataacacttcaaggatatttttactgacccagaaaaaaaaaaaggcattagatagagctactgtgactttcaaaagagaggcaaaaatgatgtcctcaaatgtgttttgttttgctagaatattgtaaaaaaaatcataattaaacatctataagtcataccctctccagagcccactccttactgtcttatgctctgcagcctcacacattaatgtgcgttaccagatcactgaaagcatcgcgtttccccacacttcattatataactcaagtacctatattgatgacactaaattcaatgctacatgcaaacatgggtgtgcctgttttcttttaatatctttaagtaaacgaggaatttttaatcacttttaaaaattgttttccctttgaaagcttcaacaagcattccaaatgttactgagatgaaaaccaacatgagcctcggcttgatcctcaccaggaacatgggaactggaggttttctcgtgagtgtttactttacaaatcattattcctctcaagaaagtacatagacagtagtgtcttctcaccatccctcccaatctgaggatttagatgtttagaacttgctttttgataaattatgctatttatttggctatccaatgttgaatattcaaaataatgttgatgtgtagactttttaggatattcagcaaaaccactgctactgaatgttttgaagaaaagggaacaaaaagaaccctgattctttgcccttggaaagtgtgctgagtatttgttgataacagcagaatcccaaatctcatttctacttcctgtaggatcattcaagatagtttcaaagctaaaaagtatttttggtttttaatcatttcctgtaattgttgagtaatctggtgttatctgaggcatggagcagtctctcatgtcagtgctcactttcatttagttacaaaaagagtaaaaagacatcactaatcagtgaaatagaatgccttatttaacaagtgcttttggaacaattaattgattaatatttacatatccaaatacactgtcaagtgaattgaaagttaatataaaaaaactagaaaaaaatggaaattttctgaaggatggaaaaatttctgagtttagagctaatagaaaaaataatataggaaattgttgaaaggatttgactatataaaaaaagggaaaaacatcacacaccaaaaaaaaagtttgaagacaaaaacacaatcttatttgaaatttccattgactcatataggtatgtgtatgtatgtataaccctgttaaaattgtcttagagaggttgggcgcggtggctcatggctgtaatcccagcactgtgggaggctgaggcaggtggatcacatgtggtcaggatttcgagaccagcctggccaacatggtgaaaccccatctctcctaaaaatgcaaaaatcagccaggagtggtggcacacacctgtaatcccagctactcgggaggctgaggcaggagaatcactgggacccaggaggcaaaagttgcatgcactgagccgagatcgtgccactgcactccagcctaagcgacagagtgagactctgtctcaaaaaaaaaaaaaaaaaaaaagaaaaaaaagtcttagacagcataattcaactttacactagttttatctgaaccattggcacgctttcttaaaaatcctatgttttataactgtattattctaaaaatgtaatcaattttcaacgtcattctatttttactaatggatattattattctgatgactgtgctgccagtaagtgaattagatattgcttgtgatatgtgttaacatcagtgcataaatgaatattgcaatggatctttatggtttatgatattccaaggtctttacaataaggttgctttaatctttttgttttttgattttaaagttgcaaaaatttaaaaaaagaacatactgatatttagagattacagaatgtgcctcaaaatttttaatctataactccagataaagtaaaaaatgaaaggtaaaaagcaagagaatttgagagattaattagcagtccaggtagcaagcagagaggcagaaagagagagaggaggagaaagaaaaaaagaaaaaacccaaagtttcctttacatacttagaaggtcaaaatcataagatactcttaagaattatgtggaaaagactgacaatcatcacatttttttgaaacttcttaagttctgggcttaaagtaggaatgtagttctggcacttacaattggcatattttaatttcaacttcttaatgctgttttccattgcttttaaaacaaaaaaaaattttgaatacttcatgagttctttttcaaagtgattttaaaaaaagcttaggaatggggatatagaaaccaataattagtatctgttatttggtaaactaaccaaagacattaataatgactttcttctaattaattggcaaaataaatcagttttatagggagaaaactatgccttgttcattgcacatgaaatatggccaaagctgcttcccctttgacagttaaactgggtcaaataagttttcttttatttttccaattacttcttgcaagcccatactcttctgttcagttttataactgattcacaactttacataacgttaaattctgtaggcattttttaggatcttaattagatttctcttttgcacttgtctcctttttcatgtatcaattttaaagttatttaatctaatgtttcttgtgtcatttaatcatgatttaatcaggagtttttggcattcaaggactcctgaaaaggagagaattgggattgcattaatggagatattaaggattggagcaggtataatgtgagcaagaagacaagtgccattcagcgttagtgactgcagttaattgggaagtggccaaagttaaaaactggtagtcagtgaacctcatttgtgtttgcttagcatatggagtaattttgaactgaattttcacacaacatttgccctctcgactctcttttacaaattagggaatctggcaacacaacctatttcatacccgtgggcaggcagaaaacatagtggtctgtacataggcatgggttcttcattcaccatgatgtctatacctgaccccaagccctcttcaagcgttactgccattaggtttatggtttctgctgagcagtatgaatggaaggaactgcagaaaactaatgttcagcaagaacaatgataatgatgctagctaacaatattatatggcatttgctatttacctggcattgttcttagtgttttgtatctatagacccatttaatctttacaacaatcttaaaagatagaatgtgtggttatcctcattttacagatgaatcacctgaggcccaggtaaatgagagacaaaaagtgaacctgcatgcattctcctccttggtgttctctggaaggaaagcacctgttttatctagcttgttgggagacctcataggctcctcccttcactgacacctgctcccaacagtctcagcactcctcctcccgcaaggcttgccactgggcacagagcacctgccaccttctccatcacttctgctttccccgaaggcattactgactcattggttttgagttgacaattattagtatcttagaaaagagtagagatgatttctgagctccataatggagaggtagaaggtttgcatttttcatgtgtttccattgattgttttctcatttctttgaagacatgtggtcctctgtgggcacagcaatgtgggaatcagtattacacaacgggtgtgtgttctgacatcagtcctgattttcagctctcagccagcttctcacctgcaactcagcgtaagttattaatgtgcagatggtctgagcaatgccttacagttaaaggagggggaaaaggttatcaactagtggcacccaaaccctccttaagtctcatttttatatggctgtgaaatatgacttacctgtggaaatctgcttctttcctcttttcctgtgtagcctgcccttccctcatagatgttgtggttgtgtgtgatgaatcaaatagtatttatccttgggatgcagtaaagaattttttggaaaaatttgtacaaggcctggatataggccccacaaagacacaggtatggctaacagaaatgcataactaacttatggctttctttctgaaaaaaatattgttagctatgaagtcttaatttttgtatttgccaaatccccattttaaatgcatttgatggtagtttttaaagttaattgaaacttttactacattgggttattctacctatggctttgcccagtgctggtcttttgaccttagagttctatttctgacacaagcattaaaggtatgggacataacttttttttttttttttaagaccaagtcttgctcttgtcacctaggctggagtgcaatggcacgatttaggctcactgcaacctctgcctcctgggttcaagcgattcttttgcctcagcctcccgagtggctggtattacaggctcctgccaccacacccagctaatttttgtatttttagtagaaacaggatttcaccatgttggccaggctggccttgaactcctgacctcaggtgatccacctgcctcagcctcccaaagtgctgggattacaggcatgagccaccatgcccggccaggatataacattttaatcttcctttagtttcctgtctttcccttctaacccatgagttacattttaaaattcaatatgtatttttatgtccatttggaccaataattaatttcttttgtttattccttgctggtaaaacaattattgaattctgaaatttgtataaataagtcactaagcatagttccagcatttatgataatattttatagttatttatcatattccagtttagcttgcccttttgcctacagaaaaggtcttttcaataattttatatagttctttatattttaagaagtctttaatttgcttttacatcttcttgaggtaggcacctaaagctcaatagtattattttaaagttcagtttctggttttggattttttttttcctttctgattgaatctgccatttcctgagccattctggttgtagcagtatactgaaccagtgtcaacccggaccagttcactgttaccagtgtatgctcatggattgcatcttatactctaatgtccaacatctaatacaaattgttgacatatttttccctaaatgctttccagctatctttattgaaatttgcttctctgctttcctgttcactattataacatgttgttatatgtttctcccattgaaattgcttttcagtaccaagagtatctcaatacccacagaagctttggaaatttccctgtgggcattttccaggacattccgctaaataaaactacatatagcactggtttccagaaagcaccactcttaaagccccttataagttttcatatgtatctccttatttcctctgtttctaaatgagttaccctcataatgtgactaattttatttaagtctcctatatagaattcacagtcttcctcttttgtgtcttcctaccacagtgcatctgattcctctctagaagtggtttaactcaatctcttttcttacagcctaaatttgaccttcctgtaagttctaacctaccacatttctttaaatcttttttcttctttttcacacagaagtcctggggctctgtcgtgggattactaccatcttcctctcgtattggactttgaaattctcagttattttttattctttcctccctctcaagtcttacatccctagagaaaggggtccaggaatattatatttctcactatcttcccagtgcctagctcactgctcccttggcaaaacacagcagatggaactcagaaatccagtcctcaccgcttccacctccttacatactgacctgtatttcagagagcctcttattgccattcagagatccctcactgccactcctgttctggaaagcccatctcctaggcttagaggatgatctcaatgcctgcaaacaaggacattaacagaagggtgtgaccttattgatctctaggattagggaatgctcccaaacatcttgttcagaaattctttcaaataagatgctctccagtggcattttctctgaaagccagaaactccctgccccatatgcacatatagcatcttctttccagtgttgttgaagcccataatagaggaaattcagcacaataaatagacctcaattgactacgctgattaggagcacattggtttttgttgtattcaattctaattatttattacacagttaccatcctttgatgagatgttactcttcatctgtgattgcttatagttgttcgcgagcttctgtccattggtaattagaaagtttatttatatcaagtttaatcttcctgttaaaaacagtgttctaatagtcatccatattaaaatattatatggcagtattaaaaactacaaatattactcttgggaatcaaatcatacactgtagcacatcatctttcttggcaatagtactgctgttgtacactgatggcctctaacagagaagaaatcattccattgaaagaaaagtaactatcaagaacaaagttggaagtgatgccttaaagctaccggcccatgtctaaatgtacttttgatttttattttattggttaagtagaaattatttttaatgtaatgacagcccattaataaatgtctcctctgttgaaggtggggttaattcagtatgccaataatccaagagttgtgtttaacttgaacacatataaaaccaaagaagaaatgattgtagcaacatcccagacatcccaatatggtggggacctcacaaacacattcggagcaattcaatatgcaaggtaagttttggtgctaataggccaatgttttcataatgtaaaacattatatttatgtaataaatatgaaaaagtaaggaaaagacaaagaaaaataatatacctggtacctaatttaaatcagaactaataaagaaaaaaacatcagagcattctatgtcttgaatactttgagaaggcagctgggaaagttaaatctttgattttaggatatttataagatatcacatgatatttaaatgaatttatgtgaagtaaatgaaatgagaagaccttagattaaaacagtaggaaatggggcaatctgtcataatttgttaatattcatcagagattcagacaaattgagctcatggatcacttggtgcaaattaacaaagaccacagaatcttaaacatacgagtttaagtttattatgcttattacaatcagtgggaaatgtgaaattataactatacaggactctcttgattcttatctcaatttctctattttctcagatccactgacctttcttttcactctattttttattttatttttattatactttaagttctgggatacatgtgcagaacgtgcaggttcattacatagtatacacatgccatggtgatttgctgcacccatcaacccatatctacattaggtacttctcctaatgctatccctctcctagtcccccaccccttgacaggccccagtgtgtgatgttcccctccctgtgtcctcattgttcaatgtgctctcattgttcagctcccacttatgagtgagaacatgcagtgtttggttttctgttcctatgtaagtttgctgagaatgatggtttccagctccatccatgtccctgcaaaccagataccttcacaaaaatgctaaacacactccggttgttaaacaaattaatgagaacaacaagcttgccttcccttaaacctattctctctctttgtcttgtgcattccttctttttaccaccataattctttttgaaaaaagcaactctattccctccctctacttccttgaaacttacagttctgccactagataagcatgaccctcttcatcccccaaccctgtggtcccaccttaaacctcatcctttctggctcctctgaacatttgataatgatgaacatacgctgctattcctaaccaagttgccatgacaccacaaacttctcgaactcctgccaaattcttttgcctcttcactagctccttttcctattttttcttcactaaacagagccctctcttctttactgctcttctcctgtagggattctatccactgtcatggcatcagcagcagcactgttgggttatctcattgctatgttgtcagccctacactctctcccaaaactcacactggcatctagctgatgatgaatatttttatttggagtgttggctcacttaccaaattcaatctgtcccacactggccttattcttttccctaccactcctcatgaacttgccattttgattcatgccactgtctcttgcctagtcagtcgaactaaaaatttggtttcctttattctttccactcttccttatgtcacacatctggtcagtggccacatcctgtcatttctatttctgcatttcccattttatctgtcttctttttctgtgtgtgtatttcagttgctacttccgtcaactggtgacaatcccaaatgtctcaaaagcagattccccacaagagaagatatgattggtttggttcatcattactgtctctattcagggacaataaaggttttttgggccaggccatctcataagctgctggccagtcaataggtcagctgccattaggtgaagcgccctttctggacacagcaaactggactaaaaggacaaagaagttgagatgaaaaatggctttcctggagttagaaactgcaacggcccggttttcagcagggaaggtgggagtagcatttttccctggaaggagcctggggccctggcgagctttgttctccaggacaatggccactgtccattgaggaaggacttgcctggctttcatcatgtttcaaaactttggttttctcccttgggccattgtctatctccctcttcccactatttcccaagctaacaggttttagtctgcccagtgctaggagaatcacccaacattttactcatccctcctagaatgtaattgcctgtctctctctctctgtctgtctctcatctctttctttaaaacattgaagataatctgaaattatttcagtaattgttgccatgttttcattcacctcatttcattcaatgcctacagttgcagtttattgttttaagcactgcgggtaattaaaactaaatgagacacagtttctgtcctcaaaaaccttagaatcatacatgaaatttacagtgtatcataaagagaatgtgacactcagaaaggatctgatgtggatagtgtaattcaaatttggccgaaaaatgcagaatattttctatgtttctatagtctacattatagaatacaaaaaagattttaggaaataaaatgatatgctcagttttataataaactgaaactcatatgtttaagattctgtggcctttgttaatttgcaccaagttatccatgagctcaatttgtctgaatctctgatgaatattaacaaattatgacagattgccccatttcctactgttttaatctaaggtcttctcatttcatttacttcatataaattcatttaaacatcatgtaatatcttataaacatcctaaaatcaaagatttaactttcccgactgccttcttaaagta

SEQ ID NO: 3 - DNA, 6751 bp, Homo sapiens
ccaaataccacctgttccccaaaaacctatggaaataaaacattttttaaaaaagaatatgccctcatttgagtaaaagtttaaggggtagactatgcctgtacctatattacatctaaataaaacattttctagaaggaaatgcaagaaactgataatggtgcttacctgtggagaggggtttagtgtcacatttggtttatacctttccaaatactttgatttttttttaactgtggatatctattacttttaaaaaaataagtattgataaagtaaacacaagactaatgcataaaatttataacagaaatatctttgctttattaattcatactgattaattcaactttcttagaagaaaagaattctaaggaatactgggataaatacatgcacacatatacacacatatgccctgacacacaagacattgtttttaatgtcaagcaagacttccatttgtatatgtttctctacaagctacaaatggaacattaaatatgtcctcagaattaatatggagttacaatagattgtgtttaaagaagctagtttcatgtttctattttaaaccagaggttctcatattaacttcatattttgatagcaagtctttatttaattttatctgccacagaaaatatgcttattcagcagcttctggtgggcgacgaagtgctacgaaagtaatggtagttgtaactgacggtgaatcacatgatggttcaatgttgaaagctgtgattgatcaatgcaaccatgacaatatactgaggtttggcatagcagtaagtggcttttcttttcacttgtcttgccgctattgggtaaatctttctttatagcatcacttctcaaggctgcactttcccattggctgttcatagttgaatataaaagaaagttcttactctatctctgcttcttgatgattttcaatcctgtctactaaataaagggttatggaaatgttgcttgatttacttttccagagggaatattgtgttcaaaataggctagaatgtactcatagggaaaatgtacagcagtaatgactgcacattctcttcctctattttcaaggttcttgggtacttaaacagaaacgcccttgatactaaaaatttaataaaagaaataaaagcaatcgctagtattccaacagaaagatactttttcaatgtgtctgatgaagcagctctactagaaaaggctgggacattaggagaacaaattttcagcattgaaggtaaaaaaaataacctcctttcaagaattttcttcaaaatgtttaaataattgtttattatcacatatttgctttactaatgttaacttgtataccatgtataaaaagagctactacaatcagtaagagaaatggatgaaaaacatgaataaatagtttataaatgataaaatacaaatgactgataaacataagaaaatattcccaatttacaaagtatatttctaaatactttcagtaagtatttaaaaacataaattaaaccgacaatatgttttgcctaaaagatgacacagaataataatgtttaatcctggagagattatcaggaagcaaagaagctcatacactgttgataggatgataaattgttatgacttttgggggacaatgtatgaatatctatctattagaatcataaatagttttacctttttacactgtgttatgtaagttctaaaattcttgaatttgtttacagtaagcatatatatttttaaaattagaaaaatacagcaaagatttttaatagttcagtttttgaggaagcattttatttataggacctaaaataatttacttaggtggccttggaaaatctagttagtgcagaaagatcacgtgttcaggagctcactgtgtgggggactagcacacacttcacagttggttaaaagaaaaaaaaaacacacatcacccaaatcctagttcttgatggttaacccgaataactctttgcaataaagaccatatgttattggaaacctatgtatcaggtacagccttagagtctggggctaaaggaattgcataaattattcattcatatactcaaatcttgatttgattatccattggaattacccaaaaagcattattgcttcttcagtatcccatggtgcttcaaagttgtttaaataaccatttaaatccagcataccctgagataacacaaaatcactaggctatccacactgataattgtatttgtttgtagtggaatgaatgagaacaattgcaatctacaagctagctagaaatgattttgagcattatttgctattatggactttggggaacattattctattttattaaaaagtatctctactcatcctgtgtaaattattttatcttctaaataagactatgttgtacagtacagtttgtcttcttttctatattaaagtgtgagtccagtaatttagcttccttttgtgaagattagtgaaacgtagccattttgatcacactgtatggaaaataagtaccaattgcatcttacagtgttagtgacacaggcaagcacatatacagaaccagaacaaacttcccaattggtaaaattcctactcagtcttgaattctgattcctaatagctaactcagttatctcccagcagtcttcactacagatcaattgcccagcccttccaactgcatctgagatatgtgtacattatcagtacccatatttcaaagctaaaaattcagttagtttcttagctagtaatgatcaagtttttaaaaaattatcatctcagttttcaccttaccttataccctttgttgagtgtcctttggtatcaagaacgtcatacatattctgctaaatacattaatgttcaattaaagttaatgccaactttgaacactaccttggaagtgttgttatttccataacctgctagaacactttcttagtaatttcttcttcccagaatatgcaagctctgtatttttgggtgagtattggaacagtgtgtgtttgtgtttgtgtttgtaggcatgtgtgtacatacgtgcctgcttgtgtttgaaaacttgatttaaaattactgacagtaatacaatttttaaaattgaatgttccaggtactgttcaaggaggagacaactttcagatggaaatgtcacaagtgggattcagtgcagattactcttctcaaaatgtgcgtatatcagatagcttcaagccatgttgtcatttggcataacacttccagacacagtagccttatacataaaatgggaagacagccatctagggatcagcccttctgattcctagtcacggtcatttagtttctttgtgctgctgatttacctctaaaatttgaaggataataatgtcttcccttccctgtttcacagggatcttatatgggggaaaactgatagtataccagaaagtctttgagtaccccagggcacagactgtatattaacttaaggtattattcctgcaggaattttctttgaatcaattaactactggaaatgtttgttaaaaacacgtactcttgtcatcaccgtcatggtcgaattctgaatggtcatagtatctttttctaagagcagacagaaaagagttgaaaaaataaggaggtaggaaaaacgtgaggtggaattggaatacaacactgtcttttcagctcggggaagtctatttctatctctcagaaacatccacctcctgggcccctgattctaatttgagtcaggaggtgagagagatagtgaaaggaggcgaaaaaaaaaagaaagagatcctgggaattatttaaggaacagtaggaaatcccaggatttgatcggcaggtcagaggaccaagaacagcttgttttctatctagaatcaaaggaactgactttttctggatcatcaatccttagagatgctcatagaagttatggatctttccctcagaaaaaatgtaaatgaatctatgcacggagaattttgcacacaatcccatgaggtttttgtacatacgctgaagtcctttcatgcaccccaaatccagagtccctcaatttactacagtcattcacataacttcatggtctcttatcctcaaagtagaacacatcttttccttacttgctatttttcatggcttgtattttttagtccaacatcttatctttggtacgctctaagatagaagaacaaaaaattaaaaatagtgtatctcatcctcaccaaatctgcctcgtttgatgatttatagcattgagtgtttagattgggtttcaagtcaatccagagtaggcatcttcctttgccctccccacagggctgacaaatggttattttccttccaatagattgaatgtcacagctaaagttgcctcttgtagctgtgctattatcaaatagatggaatataggaatatactggaacatttgagaaggggtcaggaagagagaggaagaaagctgaaagtagaacaggtcagaagagggcctcccgagccgtgtctgggatagcagaagtatctccgtctctcccgtgtcagtacttctgtttctcaagcatgtctcatatccggctgtgagctttccttataaatatgtggagtgagagactcacttctgactcctttgtaaagttcataagggtttaccattggcaaatatatgcctttgaaagggaaatttgaagatggctctgtacctgtcctgtactacaggttattacaaaggtttgtctgtataacatatcatactaaataaagtccaaaataacacagccaatgggaaggcttgttctctagcacaaatatattaagacaattctacaatatgtcaatatatgttcatatatatttcatcatttttaataaatctaaacaacttatttcaatgttttgcctaccctgcattcttatgttttaaaggtgatttgtttcaatgatcttcatttttcaattattttaggatattctgatgctgggtgcagtgggagcttttggctggagtgggaccattgtccagaagacatctcatggccatttgatctttcctaaacaagcctttgaccaaattctgcaggacagaaatcacagttcatatttaggtaaggcatggtaataattggctcagcaaacttaagttcccttgctttaaaccagctctttgttgaatctcagggtgagttcagcaaaatccttgaactgtcatctaattatcatatttattacacatacatatgttgtctctttgccaatcgggccttaaggcataactacttattggaatactatattatactcactagtttcttctggctggtcaagtggttgtaattcctggaacatgattgtgttcctccaaaatggaaatagttcccaaaagtcaatacagtaagtgatagtcttatagtattttagagctcttagtcttcttgttcttataataagcagccactgattttgcagacttactatgggacagttcctgagctgctagtctatctacataaacatttaaatctttccaacaagtctttcagggatgttttgcctttgagagatagcaagattaacaacttgtctaacgttcaacaacctagtaagcggcaaatccagaatctgaacctcagcctgtttccaagacctgtgttctttctactgtgcaacactaccttttaaatacatttattcattcgttcaagaaatattttttgaatgccaggttcacaggtggctgatgagaacagcgtggaatctagaggcagttttataggtagaagtcccacctgcaaagggaagtgctgcaggtcaaaggacaggagcaattcatcttagaaaaaggaagacatgtcctcagtgatgtattcaggagcactagaaactttgcatcacatctaacacaaaaggatctctggtcacctttactcacttcttttaactttattttgctcagatagtcaatgttaatcactctgttgctccttcccttttaggttactctgtggctgcaatttctactggagaaagcactcactttgttgctggtgctcctcgggcaaattataccggccagatagtgctatatagtgtgaatgagaatggcaatatcacggttattcaggctcaccgaggtgaccaggtaaatctcactgtttagcaggtgaaattaattttaggggcaactgggcaggtggggagtcaggtgaacagaaatggaaaacaacatggcctgagtgcctactctgtgattggctctgtaatctgagtttacttagtgtattccttaaatcatcacaacaataccaagaagacagccctcccttttgacaacttactgggaatttagctagctttctaaggtcacacagcgtccatggattgagctgtcattaaaactcagtcctttctggactcagaggctgtcctgaatgattacatccccttcatgtttttgactccctggattaataatgcatggatattatttagttgtgatggcaggcatgtaaccactgctttgtaggtaagcacacactaggaacaaggatttggagaggaaataatgagaagaagggagcagcacaacattataaggactgaacttttacatctgaacctgtcagaatgtcctgttggttttgaagattctaaaagtaaaacaattatcttctctgccactttcagctgatgggtgacagtttggtagttagaccctatgatgaatcccagatatggcccact

SEQ ID NO: 4 - DNA, 24 bp, Artificial Sequence
  Synthetic primer
gatttaactttcccgactgccttc

SEQ ID NO: 5 - DNA, 24 bp, Artificial Sequence
  Synthetic primer
cataggtttttggggaacaggtgg

SEQ ID NO: 6 - protein, 3 aa, Artificial Sequence
  Synthetic peptide
RKK

SEQ ID NO: 7 - protein, 4 aa, Artificial Sequence
  Synthetic peptide
RKKH

SEQ ID NO: 8 - protein, 6 aa, Artificial Sequence
  Synthetic peptide
CRKKHC

SEQ ID NO: 9 - protein, 7 aa, Artificial Sequence
  Synthetic peptide
CTRKKHC

SEQ ID NO: 10 - protein, 8 aa, Artificial Sequence
  Synthetic peptide
CTRKKHDC

SEQ ID NO: 11 - protein, 9 aa, Artificial Sequence
  Synthetic peptide
CTRKKHDNC

SEQ ID NO: 12 - protein, 10 aa, Artificial Sequence
  Synthetic peptide
CTRKKHDNAC

SEQ ID NO: 13 - protein, 11 aa, Artificial Sequence
  Synthetic peptide
CTRKKHDNAQC

(19) United States
(12) Patent Application Publication
(10) Pub. No.: US 2011/0150859 A1
(43) Pub. Date: Jun. 23, 2011

(54) METHOD FOR SELECTING RESPONDERS TO BLOCKADE OF INTEGRIN RECEPTORS

(76) Inventors: Anne Marjamaki, Littoinen (FI); Liisa Nissinen, Turku (FI)

(21) Appl. No.: 12/596,292

(22) PCT Filed: Apr. 16, 2008
(86) PCT No.: PCT/FI08/50195

§ 371 Date: Oct. 16, 2009

Related U.S. Application Data
(60) Provisional application No. 60/907,781, filed on Apr. 17, 2007.

(30) Foreign Application Priority Data
Apr. 17, 2007 (FI) 20075258

(51) Int. Cl. A61K 39/395; A61K 38/16; A61K 31/18; C12Q 1/68; A61P 7/02

(52) U.S. Cl. 424/130.1; 514/13.7; 514/601; 435/6.12

(57) ABSTRACT

The present invention relates to a method for identifying responders to a blockade of integrin receptors, as well as to a method for determining responsiveness to a treatment involving with said blockade in a patient in need of such treatment. Furthermore, the present invention relates to a kit for use in said methods.

### FIELD OF THE INVENTION

The present invention relates to a method for selecting responders to blockade of α2β1 integrin receptors for clinical studies, as well as to a method for determining responsiveness to a treatment involving the blockade of α2β1 integrin receptors in a patient in need of such treatment. Furthermore, the present invention relates to a kit for use in said methods.

### BACKGROUND OF THE INVENTION

interactions between cells and collagen are necessary for many physiological functions including cell migration and differentiation. The same interactions may, however, also promote mechanisms associated with diseases, such as thrombus formation and cancer spread. Cells, including blood platelets, bind to collagen fibers through specific collagen receptors called integrins. The endothelial cell layer of the healthy arterial wall prevents interaction of circulating blood platelets with collagen. At the site of endothelial damage, collagen is exposed and platelets adhere to the damaged endothelial surface initiating a cascade of events leading to thrombus formation. Adhesion of blood platelets to collagen is mainly mediated by the collagen receptor α2β1 integrin. It plays a key role in mediating firm adhesion of platelets from flowing blood.

Epidemiological studies have demonstrated the role of α2β1 integrin in the development of thrombosis, and high expression level of α2β1 integrin on the platelet surface has been shown to be an independent significant risk factor for thromboembolic conditions (Kunicki et al., Arterioscler. Thromb. Vase. Biol., 2002, 22:14-20). In addition, high α2β1 integrin expression has been connected to acute coronary syndrome including myocardial infarction. The expression level of α2β1 is defined by six different α2 alleles, and certain specific polymorphisms affecting the expression level have been identified. One particular polymorphism having an influence on the expression level is a single nucleotide polymorphism (SNP) C/T located at the base pair 807 of α2 integrin cDNA. T₈₀₇ has been associated with a higher expression of the receptor α2β1 on platelets and C₈₀₇ with a lower expression density (Kunicki et al., Blood, 1997, 89:1939-1943). In normal population there is high individual variation in the level of α2 expression.

The SNP T₈₀₇ has been suggested as a significant risk factor for thromboembolic diseases. In particular, the SNP T₈₀₇ has been associated with a risk for cerebro-vascular stroke among young patients (50 years) and diabetic retinopathy. However, it should be noted that many other studies have failed to show such connections (reviewed in Kunicki et al., Arterioscler. Thromb. Vasc. Biol., 2002, 22:14-20).

Thus, high expression level and genetic variants of α2β1 integrin seem potential risk factors for thrombotic diseases but their role in response to treatment is less clear, and reports on the role of α2 polymorphisms in platelet sensitivity to the blockade of α2β1 integrin receptors are scanty. Rozalski et al. (Pharmacol. Rep., 2005, 57:1-13) discloses that subjects having a TT₈₀₇ genotype respond to treatment with a monoclonal anti-α2β1 integrin antibody better than subjects having a CC₈₀₇ genotype. This report, however, is silent about responsiveness of subjects having a heterozygous CT₈₀₇ genotype.

Identification and selection of subjects who respond to the blockade of α2β1 integrin receptors in general would significantly improve probability of success and drastically reduce the development costs of novel α2β1 integrin inhibitors for clinical use. Furthermore, determining responsiveness to a treatment involving the blockade of α2β1 integrin receptors avoids unethically treating patients known not to respond to the treatment. There is thus a need in the art for a method for selecting patients who respond to a treatment involving the blockade of α2β1 integrin receptors. Such patients would benefit from the treatment with inhibitors of said integrin receptors in preventing, treating and/or alleviating thromboembolic conditions, and should be selected for clinical studies aiming at developing novel antithrombotic drugs.

### BRIEF DESCRIPTION OF THE INVENTION

The present invention is based on a surprising finding that individuals having an α2 integrin genotype TT₈₀₇, as well as individuals having an α2 integrin genotype CT₈₀₇ combined with a high overall expression level of α2β1 integrin, respond to the blockade of α2β1 receptors.

The present invention thus relates to a method for identifying a subject responding to the blockade of an α2β1 integrin receptor. Said method comprises the following steps: a) determining a single nucleotide polymorphism (SNP) C/T located at the base pair 807 of α2 integrin cDNA depicted in SEQ ID NO. 1, in a sample obtained from said subject, b) measuring the expression level of α2β1 integrin on platelets obtained from a subject having a CT₈₀₇ genotype, and c) identifying as a subject responding to the blockade of α2β1 integrin receptors, a subject having either a TT₈₀₇ genotype, or having a CT₈₀₇ genotype combined with a high expression level of α2β1 integrin.

The present invention further relates to a method for determining responsiveness to a treatment involving blockade of α2β1 integrin receptors in a patient in need of such treatment. Said method comprises the steps of: a) determining a SNP C/T located at the base pair 807 of α2 integrin cDNA depicted in SEQ ID NO. 1, in a sample obtained from said patient, b) measuring the expression level of α2β1 integrin on platelets in vitro, when said patient is determined to have a CT₈₀₇ genotype, and c) designating as indicating responsiveness to the blockade of α2β1 integrin receptors, a patient having either a TT₈₀₇ genotype or a CT₈₀₇ genotype combined with a high expression level of α2β1 integrin.

The present invention further relates to a method for treating, preventing and/or alleviating a thromboembolic condition in a patient, said method comprising administering to a patient having either a TT₈₀₇ genotype or a CT₈₀₇ genotype combined with a high expression level of α2β1 integrin, an effective amount of an α2β1 integrin inhibitor.

Furthermore, the present invention relates to use of an α2β1 integrin inhibitor or a combination thereof for the manufacture of a pharmaceutical composition for preventing, treating and/or alleviating a thromboembolic condition in a patient diagnosed as responsive by having either a TT₈₀₇ genotype or a CT₈₀₇ genotype combined with a high expression level of α2β1 integrin.

According to some embodiments of the present invention, the patient suffers from a thromboembolic condition, such as angina pectoris (including but not being limited to unstable angina, stenocardia and unspecified angina); acute myocardial infarction; subsequent myocardial infarction (including recurrent myocardial infarction); thrombotic and thromboembolic complications of myocardial infarction; other ischaemic heart disease (including but not being limited to coronary thrombosis not resulting in myocardial infarction); chronic ischaemic heart disease; pulmonary embolism; cerebral infarction (including but not being limited to infarction due to thrombosis, embolism, occlusion or stenosis of precerebral or cerebral arteries, or cerebral venous thrombosis); thrombosis, embolism, occlusion or stenosis of precerebral or cerebral arteries not resulting in cerebral infarction; transient cerebral ischaemic attacks and related syndromes; thrombosis of intracranial venous system; arterial embolism and thrombosis (including but not being limited to embolism and thrombosis of arterial aneurysm); thrombophlebitis; portal vein thrombosis; other venous embolism and thrombosis; thrombosis of autograft, allograft, xenograft or prosthesis; posttraumatic thrombosis (including but not being limited to complications of intravascular procedures such as stenting or angioplasty); and thrombosis of a surgical anastomosis.

The present invention further relates to a kit for determining responsiveness to the blockade of α2β1 integrin receptors in a human subject. The kit comprises: a) PCR primers and, optionally, other PCR reagents for amplification of α2 integrin gene, b) Bgl II restriction enzyme and a suitable buffer, c) an α2β1 integrin binding reagent for detecting the expression level of α2β1 integrin on platelets, and d) instructions for determining whether said human subject is responsive to a treatment involving the blockade of α2β1 integrin receptors.

According to some embodiments of the present invention, said blockade of α2β1 integrin receptors is achieved with α2β1 integrin inhibitors or α2β1 integrin binding reagents including antibodies, compounds such as sulphonamide derivatives, or peptides such as peptides comprising an amino acid sequence RKK.

### BRIEF DESCRIPTION OF THE DRAWINGS

In the following the invention will be described in greater detail by means of preferred embodiments with reference to the attached drawings, in which

FIG. 1A shows that the donors having either TT₈₀₇ (open circle) or CT₈₀₇ (open triangle) genotype both have higher average expression of α2 integrin on their platelet surfaces than the donors having CC₈₀₇ (filled square) genotype. Y-axis represents the amount of α2 integrin in mean fluorescence (MEF) on platelets; **p<0.005.

FIG. 1B demonstrates the method by which the genotypic groups are screened. The amplified fragment is treated with Bgl II restriction enzyme and run into 1.5% agarose gel. The TT₈₀₇ genotype is indicated as (+)/(+), TC₈₀₇ as (+)/(−), and CC₈₀₇ as (−)/(−).

FIG. 1C is a schematic representation of FIG. 1B.

FIG. 2 shows that a blockade of integrin receptors increases the blood closure time in the donors having high expression levels of α2 integrin (genotypes TT₈₀₇ and CT₈₀₇). The closure time was analyzed with PFA-100 (platelet function analyzer) in the absence (Control) and presence of α2 μl integrin inhibitor (BTT-3016). In FIG. 2A the effect of BTT-3016 on whole blood closure time is shown with the responding donors having the TT₈₀₇ or TC₈₀₇ genotype and high α2 integrin levels (MEF≧35). In FIG. 2B the effect of BTT-3016 on blood closure time is shown with the non-responsive donors having the CC₈₀₇ genotype. In FIGS. 2A and 2B y-axis represents the whole blood closure time in seconds determined by PFA-100.

FIG. 3 shows that EDTA inhibits the binding of a C^{u}-labeled integrin inhibitor to CHO-α2 cells but not to CHO-wild type (wt) cells. This indicates that the labeled compound specifically detects the amount of α2 integrin on cell surface. Y-axis represents binding of C¹⁴-labeled integrin inhibitor to the cells.

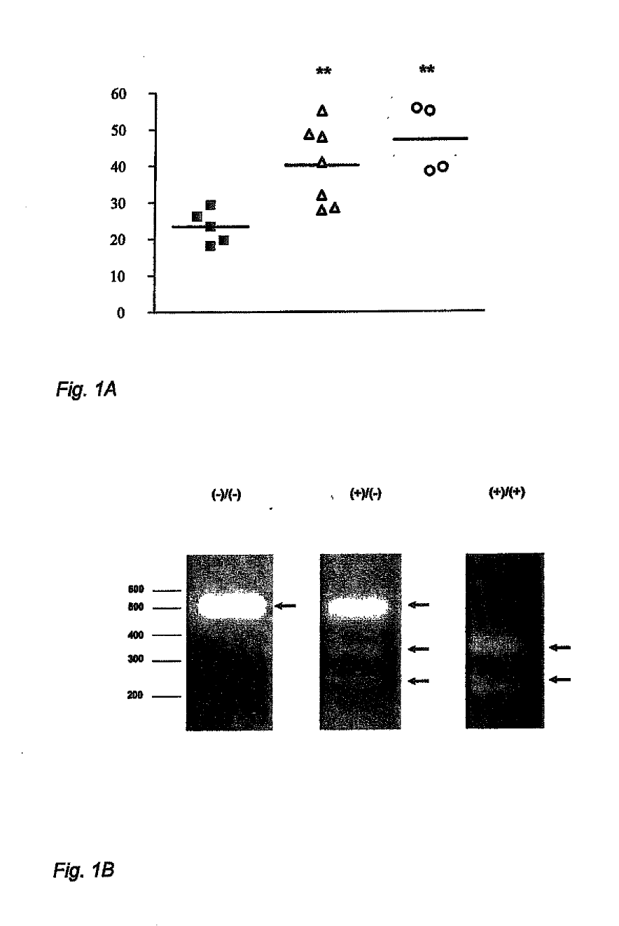

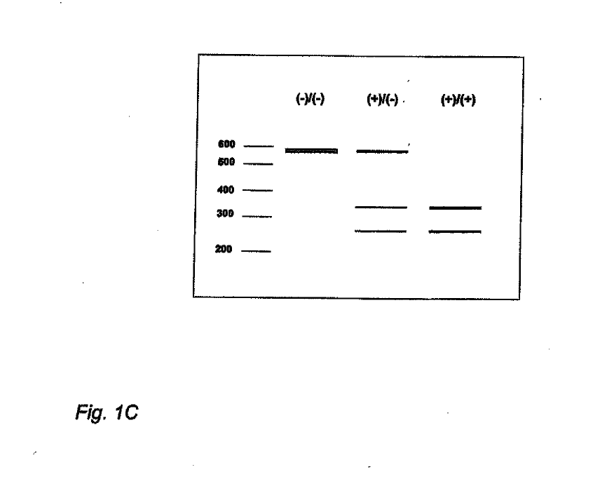

### DETAILED DESCRIPTION OF THE INVENTION

The present invention is based on studies, which attempted to find a method for identifying subjects who respond to a blockade of α2β1 integrin receptors.

Thirty (30) randomly chosen healthy volunteers were genotyped for a single nucleotide polymorphism (SNP) C/T located at the base pair 807 of α2 integrin cDNA. Of the subjects studied, 43% had CC₈₀₇ genotype, 37% had CT₈₀₇ genotype, and 20% had TT₈₀₇ genotype.

The term “CT₈₀₇ genotype” refers herein to a heterozygous genotype having SNP C at the base pair 807 in the cDNA of one α2 integrin allele and SNP T at the base pair 807 in the cDNA of the other α2 integrin allele. Accordingly, the term “CC₈₀₇ genotype” refers to a homozygous genotype having SNP C at the base pair 807 in the cDNA of both α2 integrin alleles, while the term “TT₈₀₇ genotype” refers to a homozygous genotype having SNP T at the base pair 807 in the cDNA of both α2 integrin alleles. The base pair 807 refers to a nucleotide 807 of the α2 integrin nucleotide sequence as disclosed e.g. by Kunicki et al., 1997 (ibid.) and by Kritzik et al., 1998 (Blood, 92:2382-2388), and depicted in SEQ ID NO. 1. The cDNA sequence of α2 integrin is also publicly available under GenBank accession number X17033.

The responsiveness of the genotyped healthy volunteers to a treatment involving the blockade of α2β1 integrin receptors was assessed by measuring the effect of synthetic α2β1 integrin inhibitors, such as sulphonamide derivatives disclosed e.g. in WO 2005/090298, on whole blood closure time in PFA-100 platelet function analysis. The PFA-100 analyzer is a high shear-inducing device that simulates primary haemostasis after injury of a small vessel. The system comprises a test-cartridge containing a biologically active membrane coated with collagen plus adenosine diphosphate (ADP). An anticoaculated whole blood sample is run through a capillary under a constant vacuum. The platelet agonists on the membrane (i.e. ADP and collagen), and the high shear rate results in activation of platelet aggregation, leading to occlusion of the aperture with a stable platelet plug. The time required to obtain full occlusion of the aperture is designated as the “closure time”. Synthetic α2β1 integrin inhibitor was added to the whole blood sample obtained from each healthy volunteer, and the closure time was measured with PFA-100. If the closure time was increased when compared to the control sample (untreated sample from the same individual) subject was regarded as a subject responsive to the blockade of α2β1 integrin receptors.

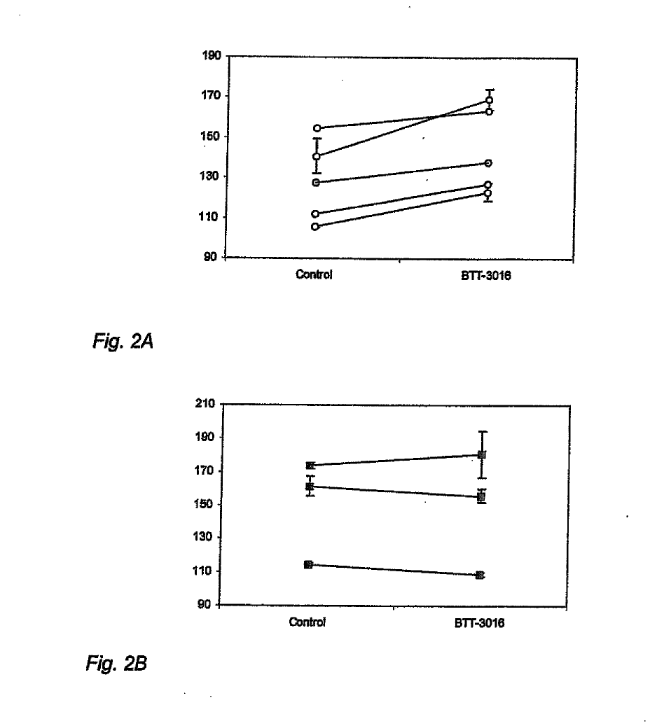

As shown in FIG. 2B, it was found that subjects having the CC₈₀₇ genotype do not respond to the treatment with sulphonamide derivatives in PEA-100 analysis, and therefore, they should be regarded as subjects not responding to the blockade of α2β1 integrin receptors.

On the other hand, all subjects having the TT₈₀₇ genotype, as well as subjects having the CT₈₀₇ genotype in combination with a high expression level of α2β1 integrin (mean flurescence or MEF≦35) responded statistically significantly to the treatment with sulphonamide derivatives (p<0.05; FIG. 2A) indicating that such subjects should be regarded as subjects responding to the blockade of α2β1 integrin receptors.

Based on the above, the present invention provides a method for identifying subjects responding to the blockade of α2β1 receptors, said method comprising the following steps: determining a SNP C/T located at base pair 807 of α2 integrin cDNA in a sample obtained from said subject, measuring the expression level of α2β1 integrin on platelets obtained from a subject having a CT₈₀₇ genotype, and identifying a subject having either a TT₈₀₇ genotype or a subject having CT₈₀₇ genotype combined with a high expression level of α2β1 integrin as a subject responding to the blockade of α2β1 integrin receptors.

The α2 integrin genotype can be determined in any suitable biological sample by any suitable method known in the art, such as restriction fragment length polymorphism (RFLP). In one embodiment according to the present invention, the α2 integrin genotype is determined by isolating the total cellular DNA from the whole blood and performing PCR with specific α2 integrin primers. The amplified α2 fragment is then digested with a restriction enzyme, such as Bgl II, and the result of the digestion reaction is detected on an agarose gel. If the amplified fragment contains a recognition site for said restriction enzyme, it will be digested into two smaller fragments. For example, nucleotide T in the by 807 on integrin α2 cDNA creates a restriction site for Bgl II, while nucleotide C in the same position does not. Thus, α2 integrin genotype can be determined e.g. by digestion with Bgl II enzyme.

The expression level of α2β1 integrin can be determined by any suitable direct or indirect method known in the art. In one embodiment according to the present invention, platelets are isolated from the whole blood, and labelled with a specific fluorescent α2 integrin antibody. The α2 antibody staining is then analyzed e.g. by a flow cytometer. In another embodiment according to the present invention, radio- or fluorescent labelled integrin inhibitiory compound is used for determining the expression level of α2β1 integrin. Said integrin inhibitory compound, such as a sulphonamide derivative (e.g hydrochloride salt of [4-(dimethylamino)phenyl]{[3-(4-fluorophenyl)phenyl]-sulfonyl}methylamine; or sodium salt of 4-({[3-(4-fluorophenyl)phenyl]sulfonyl}-amino)phenyl phenyl ketone), binds to α2β1 integrin on e.g. platelet surfaces, and the level of binding is proportional to the level α2β1 integrin expression. Said binding can be detected e.g. by a flow cytometer, a scintillation counter or by autoradiography depending on the nature of the label in question, as well known to a person skilled in the art. In a further embodiment according to the present invention, biotinylated (e.g. steptavidin coupled biotin) integrin inhibiting peptides are used for assessing the expression level of α2β1 integrin. Binding of said peptides to α2β1 integrin is proportional to the level α2β1 integrin expression, and can be detected e.g. by chemiluminescence. Other suitable labels, such as fluorescent, luminescent, chromogenic, fotometric and radioactive labels, for labelling integrin-inhibiting peptides are readily available in the art.

When flow cytometer is used to determine the expression level of α2 μl integrin, the intensity of the fluorescence (produced by fluorescent antibody or fluorescent labelled integrin inhibitiory compound) indicates the amount of α2 integrin on single platelet. Mean fluorescence (MEF) is an average fluorescence intensity from the sample In which 10000 platelets are counted by flow cytometer. Statistical analysis of the histogram data produced by flow cytometer gives the MEF value to each sample. These MEF values of different samples can be compared to each other when the flow cytometric runs are done with the same instrument settings. The term “high expression level”, as used herein, means MEF 35.

In some embodiments according to the present invention, “high expression level” is determined by a significant, preferably statistically significant, increase in the binding of said labeled compound or peptide to platelets as compared to control platelets. A person skilled in the art knows when the difference in binding is significant, and appreciates suitable methods for detecting said difference.

The method for identifying subjects responding to the blockade of α2β1 receptors according to the present invention is useful e.g. in selecting subjects for clinical studies aiming at developing novel antithrombotic drugs. Identification and selection of subjects who respond to the blockade of α2β1 integrin receptors in general would significantly improve probability of success and drastically reduce the development costs of novel α2β1 integrin inhibitors for clinical use. If only subjects having the TT₈₀₇ genotype were included in such clinical trials, only 15-20% of volunteers could be selected. This aspect is particularly important in large clinical trials where thousands of patients are required. Now that also subjects having the CT₈₀₇ genotype combined with a high expression level of α2β1 integrin can be selected for such clinical studies, the number of suitable individuals is increased to about 40% of the volunteers.

The present invention also provides a method for determining responsiveness to treatment involving the blockade of α2β1 integrin receptors in a patient in need of such treatment and avoids unethically treating patients known not to respond to the treatment. Said method comprises the steps of: determining a SNP C/T located at base pair 807 of α2 integrin cDNA in a sample obtained from said patient, measuring the expression level of α2 μl integrin on platelets in vitro, when said patient is determined to have a CT₈₀₇ genotype, and designating as indicating responsiveness to the blockade of an α2β1 integrin receptors, a patient having either a TT₈₀₇ genotype or a CT₈₀₇ genotype combined with a high expression level of α2β1 integrin. In some embodiments according to the present invention, said patient suffers from a thromboembolic condition.

Determining the genotype and measuring the expression level of α2β1 integrin on platelet surfaces can be performed by any suitable method as described above.

The method for determining responsiveness to a treatment involving the blockade of α2β1 integrin receptors according to the present invention is useful for selecting patients who would benefit from the treatment with α2β1 integrin inhibitors in preventing or treating e.g. thromboembolic conditions.

The present invention further provides a method for treating, preventing or alleviating a thromboembolic condition in a patient, said method comprising administering to said patient having either a TT₈₀₇ genotype or a CT₈₀₇ genotype combined with a high expression level of α2β1 integrin, an effective amount of an α2β1 integrin inhibitor.

The present invention further relates a use of α2β1 integrin inhibitors or a combination thereof for the manufacture of a pharmaceutical composition for preventing, treating and/or alleviating a thromboembolic condition in a patient diagnosed as responsive by having either a TT₈₀₇ genotype or a CT₈₀₇ genotype combined with a high expression level of α2β1 integrin.

The term “thromboembolic condition” as used herein, includes, but is not limited to, angina pectoris (e.g. unstable angina, stenocardia and unspecified angina); acute myocardial infarction; subsequent myocardial infarction (including recurrent myocardial infarction); thrombotic and thromboembolic complications of myocardial infarction; other ischaemic heart disease (e.g. coronary thrombosis not resulting in myocardial infarction); chronic ischaemic heart disease; pulmonary embolism; cerebral infarction (e.g. infarction due to thrombosis, embolism, occlusion or stenosis of precerebral or cerebral arteries, or cerebral venous thrombosis); thrombosis, embolism, occlusion or stenosis of precerebral or cerebral arteries not resulting in cerebral infarction; transient cerebral ischaemic attacks and related syndromes; thrombosis of intracranial venous system; arterial embolism and thrombosis (e.g. embolism and thrombosis of arterial aneurysm); thrombophlebitis; portal vein thrombosis; other venous embolism and thrombosis; thrombosis of autograft, allograft, xenograft or prosthesis; posttraumatic thrombosis (e.g. complications of intravascular procedures such as stenting or angioplasty); and thrombosis of a surgical anastomosis.

Suitable compounds resulting in the blockade of α2β1 integrin receptors in connection with the present invention include any compound, which partly or completely inhibit or block the function of α2β1 integrin, such as an antibody, a sulphonamide derivative and α2β1 integrin binding peptides. Still another object of the present invention is to provide a suitably labeled α2β1 integrin inhibitor for use in measuring the level of α2β1 integrin expression on a platelet as described above. Suitable labels are known to a person skilled in the art including but not limited to radiolabels, fluorescence labels and biotin labels.

The present invention also provides a kit for determining responsiveness to the blockade of α2β1 integrin receptors in a human subject, said kit comprising

a) PCR primers and, optionally, other PCR reagents for amplification of α2 integrin gene,

b) Bgl II restriction enzyme and a suitable buffer,

c) an α2β1 integrin binding reagent for detecting the expression level of α2β1 integrin on platelets, and

d) instructions for determining whether said human subject is responsive to a treatment involving the blockade of α2β1 integrin receptors.

PCR primers suitable for use in the present invention include first primers, which hybridise to the region 34400-64910 nt of α2 integrin gene and second primers, which hybridise to the region 65430-72180 nt of integrin α2 gene. The nucleotide sequence of α2 integrin gene is publicly available under GenBank accession number NT_{—}006713. The nucleotide region 34400-64910 corresponds to nucleotides 1-30511 depicted in SEQ ID NO. 2., while the nucleotide region 34400-64910 corresponds to nucleotides 1-6751 depicted in SEQ ID NO. 3. Typically the length of said PCR primers is 15 to 25 base pairs, preferably 20 base pairs. In one embodiment according to the present invention said first primer comprises the nucleotide sequence depicted in SEQ ID NO. 4, and said second primer comprises the nucleotide sequence depicted in SEQ ID NO. 5.

Other PCR reagents needed for the ampification of α2 integrin gene, and optionally included in the kit according to the present invention include DNA polymerase, suitable PCR buffer and deoxyribonucleotide triphosphates (dNTPs).

In one embodiment according to the present invention, said α2β1 integrin binding reagent for detecting the expression level of α2β1 integrin on platelets is an α2β1 antibody, and preferably fluorescence labelled α2β1 antibody.

In another embodiment according to the present invention, said α2β1 integrin binding reagent for detecting the expression level of α2β1 integrin on platelets is a radio- or fluorescence labelled chemical compound, such as a sulfonamide derivative described for example in WO 2005/090298, EP1258252, EPO472053, WO 03/008380. Suitable labels and methods for labelling are readily appreciated by a person skilled in the art. As an example, compounds that are suitable for C¹⁴ radiolabelling include [(2,4-dichlorophenyl)sulfonyl][4-(dimethylamino)phenyl]methylamine, 2H-benzo[3,4-d]1,3-dioxolan-5-yl[(2,4-dichlorophenyl)sulfonyl]methylamine, [4-(dimethylamino)phenyl][(3-bromophenyl)sulfonyl]methylamine, hydrochloride salt of [4-(dimethylamino)phenyl]{[3-(4-fluorophenyl)phenyl]-sulfonyl}methylamine, {[3-(4-fluorophenyl)phenyl]sulfonyl}methyl(2-methylbenzoxazol-5-yl)amine, [(2,4-dichlorophenyl)sulfonyl]{4-[(4,6-dimethylpyrimidin-2-yl)methylamino]-phenyl}methylamine, [4-(dimethylamino)phenyl]{[3-(4-fluoro-2-methyl phenyl)phenyl]sulfonyl}-methylamine, [(3-bromophenyl)sulfonyl]methyl(2-methylindol-5-yl)amine, [(2,4-dichlorophenyl)sulfonyl]methyl(1-methylindol-6-yl)amine, [(2,4-dichlorophenyl)sulfonyl]carbazol-3-ylmethylamine [(2,4-dichlorophenyl)sulfonyl](1,2-dimethylindol-5-yl)methylamine, and [(2,4-dichlorophenyl)sulfonyl]methyl(1-methylindol-5-yl)amine, whereas sodium salt of 4-({[3-(4-fluorophenyl)phenyl]sulfonyl}amino)phenyl phenyl ketone (BTT-3016) is suitable for fluorescence labelling.

In still another embodiment according to the present invention, said α2β1 integrin binding reagent for detecting the expression level of α2β1 integrin on platelets is a biotinylated cyclic peptide comprising a colinear sequence of three amino acids, arginine-lysine-lysine (RKK; SEQ ID NO 6) disclosed in WO 99/02551. Such cyclic peptides encompass e.g. peptides comprising one or more copies of the RKK sequence motif, peptides comprising the amino acid sequence RKKH (SEQ ID NO. 7), peptides comprising the amino acid sequence CRKKHC (SEQ ID NO. 8), CTRKKHC (SEQ ID NO. 9), CTRKKHDC (SEQ ID NO. 10), CTRKKHDNC (SEQ ID NO. 11), CTRKKHDNAC (SEQ ID NO. 12) and peptides comprising the amino acid sequence CTRKKHDNAQC (SEQ ID NO. 13). Said biotinylated peptides can be detected e.g. by chemiluminescence. Other suitable labels and methods for detecting are readily available to a person skilled in the art.

Optionally, the kit may also include all or some of necessary reagents required for isolation total DNA in a sample, purification of the obtained PCR product, agarose gel electrophoresis (for detecting the result of Bgl II reaction), and isolation of platelets from whole blood.

### Example 1

### Genotyping α2 Alleles and Expression of α2β1

The genotypic α2 allele distribution in 30 healthy donors was determined by isolating total DNA from whole blood with NucleoSpin Blood kit (Macherey Nagel). About 600 bp fragment of intron G in integrin α2 gene including potential Bgl II site was amplified from genomic DNA using primers: 5″ primer (base pairs 30480-30503 depicted in SEQ ID NO. 2) GATTTAACTTTCCCGACTGCCTTC (SEQ ID NO. 4) and 3″ primer (base pairs 8-31 depicted in SEQ ID NO. 3) CATAGGTTTTTGGGGAAC-AGGTGG (SEQ ID NO. 5) (Kritzik et al., 1998). PCR amplification was done using the following protocol: denaturation at 94° C. for 10 min; 2 cycles: denaturation at 94° C. for 1 min, annealing at 69° C. for 1 min, extension at 72° C. for 1 min; 2 cycles: denaturation at 94° C. for 1 min, annealing at 67° C. for 1 min, extension at 72° C. for 1 min; and 35 cycles: denaturation at 94° C. for 1 min, annealing at 65° C. for 1 min, extension at 72° C. for 1 min. PCR product was digested with Bgl II restriction entsyme (Promega) and reaction products were analyzed on 1.5% agarose gel.

The α2 genotypes of the donors were determined based on restriction fragment length polymorphism (RFLP). Nucleotide T in the by 807 on integrin α2 cDNA creates the restriction site for Bgl II but if there is nucleotide C in the by 807 no digestion will occur with Bgl II. If both alleles of the donor contain the Bgl II site there will be only two smaller fragments (˜300 bp) in agarose gel (TT₈₀₇ genotype) but if other allele is not digested there will be 600 bp band and two smaller bands in agarose gel (TC₈₀₇ genotype). If neither of the alleles does contain Bgl II site there will be only 600 bp band in the agarose gel (TT₈₀₇ genotype).

Of the donors, 43% possessed CC₈₀₇ genotype, while 37% possessed TC₈₀₇ and 20% possessed TT₈₀₇ genotype. Previously Carlsson et al. (Blood, 1999, 93:3583-3586) have studied the α2 integrin CT₈₀₇ distribution in a group that consists of 184 healthy donors and the present results were in accordance to that.

Isolation of platelets from whole blood was performed with OptiPrep reagent (Axis-Shield) according to the manufacturer's protocol. After density gradient centrifugation, platelets were labeled with an anti-α2 integrin FITC conjugated antibody (BD Pharmingen) or with a control antibody anti-mouse FITC (DACO) for 30 min in room temperature. Cell fluorescence was detected with a FACScan flow cytometer (Becton Dickinson).

The correlation between genotype and α2β1 expression was determined by flow cytometric staining of isolated platelets. The donors having integrin α2 alleles CT₈₀₇ (mean fluorescence (MEF) 40.2) and TT₈₀₇ (MEF 47.2) had higher amounts of α2 on their platelet surfaces than the donors who had α2 allele CC₈₀₇ (MEF 23.4). Even though the average α2β1 integrin expression was elevated in both in homozygote (TT₈₀₇) and heterozygote (CT₈₀₇) donor group the variation from the average expression was greater in the heterozygote group.

### Example 2

### The Effect of the Blockade of Integrin Receptors on Blood Closure Time

The effect of a synthetic α2β1 integrin inhibitor, sodium salt of 4-({[3-(4-fluorophenyl)phenyl]sulfonyl}amino)phenyl phenyl ketone (BTT-3016), on whole blood closure time in PFA-100 analysis was determined for donors representing each genotypic population. Blood was collected into tubes containing 3.2% buffered lithium heparin as anticoagulant. Whole blood samples were treated with or without integrin inhibitory compounds. Samples were incubated at room temperature for 10 min., dispensed into PFA Collagen/ADP cartridges, and closure time was determined with PFA-100 (Dade Behring).

BTT-3016 was shown to increase the closure time statistically significantly (two-way ANOVA; p<0.05) in donors having high expression of integrin α2β1 (amount of α2 integrin on platelets MEF≦35, determined by flow cytometric analysis). The effect was not dependent on the genotype (CT₈₀₇ or TT₈₀₇). BTT-3016 was not efficacious in the donors with low integrin expression (genotype CC₈₀₇). Thus combining the analysis of genetic polymorphism at base pair 807 of α2 cDNA with detection of protein level expression of α2β1 integrin with flow cytometric staining of platelets, the selection of patients who would benefit from the blockade of α2β1 receptors in treating thrombotic disorders is possible.

### Example 3

### Use of C¹⁴-Labelled Integrin Inhibitor in the Determination Of the Expression Level of α2β1 Integrin on Platelets

The binding of C¹⁴-labeled integrin inhibitory compound, hydrochloride salt of [4-(dimethylamino)phenyl]{[3-(4-fluorophenyl)phenyl]-sulfonyl}methylamine, was studied with CHO-wt cells and α2 integrin over expressing CHO cell clone (CHO-α2). CHO-wt cells have no collagen receptor integrins on their cell surface. Cells were fixed in 2% formaldehyde in PBS. After that cells were washed and suspended in reaction buffer (50 mM Tris-HCl pH 7.4, 5 mM MgCl₂). To all samples 10 μM C¹⁴-labeled inhibitory compound was added and the cells were incubated in the presence or absence of 20 mM EDTA for 1 h in +4° C. 20000 cells from each sample were loaded to GF/B glass microfibre filter (Whatman) and the samples were washed with 10 mL of the reaction buffer. The membranes were put to scintillation buffer (Optiphase HiSafe3, Perkin Elmer) and the activity was measured with the scintillation counter (Wallac 1415).

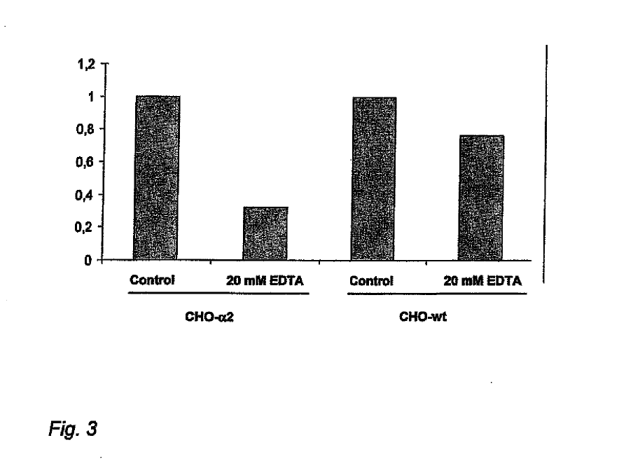

The C¹⁴-labeled integrin inhibitory compound bound more efficiently to CHO-α2 cells than to CHO-wt cells (FIG. 3). EDTA was used in the experiment because of the fact that α2 integrin needs Mg²⁺ ions to bind the compound. The binding of the labeled compound in the presence of 20 mM EDTA could be inhibited with CHO-α2 cells but not with CHO-wt cells. This indicates that the binding is specific to α2 integrin.

1-25. (canceled)

26. A method for identifying a subject belonging to a group responding to a blockade of α2β1 integrin receptors as determined by a decrease in platelet aggregation, comprising
a) determining a single nucleotide polymorphism (SNP) C/T located at base pair 807 of α2 integrin cDNA depicted in SEQ ID NO. 1, in a sample obtained from said subject,
b) measuring the expression level of α2β1 integrin on platelets obtained from a subject having a CT₈₀₇ genotype, and
c) identifying as a subject responding to the blockade of α2β1 integrin receptors, a subject having either a TT₈₀₇ genotype, or having a CT₈₀₇ genotype combined with a high expression level of α2β1 integrin,
wherein high expression level of α2β1 integrin means a statistically significant increase in said expression level as compared to the expression level of α2β1 integrin in subjects having a CC₈₀₇ genotype.

27. The method according to claim 26 wherein step a) is based on restriction fragment length polymorphism.

28. The method according to claim 26 wherein step b) is performed by a flow cytometer, a scintillation counter, by autoradiography, by chemiluminescence, by fotometry, by fluorometry, or by luminometry.

29. The method according to claim 26 wherein in step c) said high expression level means a mean fluorescence 35.

30. A method for determining responsiveness to a treatment involving blockade of α2β1 integrin receptors, as determined by a decrease in platelet aggregation, in a patient in need of such treatment, said method comprising
a) determining a SNP C/T located at base pair 807 of α2 integrin cDNA depicted in SEQ ID NO. 1, in a sample obtained from said patient,
b) measuring the expression level of α2β1 integrin on platelets in vitro, when said patient is determined to have a CT₈₀₇ genotype, and
c) designating as indicating responsiveness to the blockade of α2β1 integrin receptors, a patient having either a TT₈₀₇ genotype or a CT₈₀₇ genotype combined with a high expression level of α2β1 integrin,
wherein high expression level of α2β1 integrin means a statistically significant increase in said expression level as compared to the expression level of α2β1 integrin in subjects having a CC₈₀₇ genotype.

31. The method according to claim 30, wherein said patient suffers from a thromboembolic condition.

32. A method for treating, preventing and/or alleviating a thromboembolic condition in a patient, said method comprising administering to a patient having either a TT₈₀₇ genotype or a CT₈₀₇ genotype combined with a high expression level of α2β1 integrin, an effective amount of an α2β1 integrin inhibitor, wherein high expression level of α2β1 integrin means a statistically significant increase in said expression level as compared to the expression level of α2β1 integrin in subjects having a CC₈₀₇ genotype.

33. The method according to claim 32, wherein said inhibitor is an antibody.

34. The method according to claim 32, wherein said inhibitor is a α2β1 integrin binding compound.

35. The method according to claim 34, wherein said compound is a sulphonamide derivative.

36. The method according to claim 32, wherein said inhibitor is a peptide.

37. A kit for use in the method according to claim 26 or claim 30, said kit comprising
a) PCR primers and, optionally, other PCR reagents for amplification of α2 integrin gene,
b) Bgl II restriction enzyme and a suitable buffer,
c) an α2β1 integrin binding reagent for detecting the expression level of α2β1 integrin on platelets, and
d) instructions for determining whether said human subject is responsive to a treatment involving the blockade of α2β1 integrin receptors.

38. The kit according to claim 37, wherein a) comprises a first primer hybridising to the region 1-30511 nt of α2 integrin gene depicted in SEQ ID NO. 2, and a second primer hybridising to the region 1-6751 nt of integrin α2 gene depicted in SEQ ID NO. 3.

39. The kit according to claim 38, wherein said first primer comprises a nucleotide sequence depicted in SEQ ID NO. 4 and said second primer comprises a nucleotide sequence depicted in SEQ ID NO. 5.

40. The kit according to claim 38, wherein said α2β1 integrin binding reagent is an antibody.

41. The kit according to claim 38, wherein said α2β1 integrin binding reagent is an α2β1 integrin binding chemical compound.

42. The kit according to claim 38, wherein said chemical compound is a sulphonamide derivative.

43. The kit according to claim 42, wherein said sulphonamide derivative is selected from the group consisting of
[(2,4-dichlorophenyl)sulfonyl][4-(dimethylamino)phenyl]methylamine,
2H-benzo[3,4-d]1,3-dioxolan-5-yl[(2,4-dichlorophenyl)sulfonyl]methylamine,
[4-(dimethylamino)phenyl][(3-bromophenyl)sulfonyl]methylamine, hydrochloride salt of [4-(dimethylamino)phenyl]{[3-(4-fluorophenyl)phenyl]-sulfonyl}methylamine,
{[3-(4-fluorophenyl)phenyl]sulfonyl}methyl(2-methylbenzoxazol-5-yl)amine,
[(2,4-dichlorophenyl)sulfonyl]{4-[(4,6-dimethylpyrimidin-2-yl)methylamino]-phenyl}methylamine,
[4-(dimethylamino)phenyl]{[3-(4-fluoro-2-methylphenyl)phenyl]sulfonyl}-methylamine,
[(3-bromophenyl)sulfonyl]methyl(2-methylindol-5-yl)amine,
[(2,4-dichlorophenyl)sulfonyl]methyl(1-methylindol-6-yl)amine,
[(2,4-dichlorophenyl)sulfonyl]carbazol-3-ylmethylamine
[(2,4-dichlorophenyl)sulfonyl](1,2-dimethylindol-5-yl)methylamine, and
[(2,4-dichlorophenyl)sulfonyl]methyl(1-methylindol-5-yl)amine, and sodium salt of 4-({[3-(4-fluorophenyl)phenyl]sulfonyl}amino)phenyl phenyl ketone.

44. The kit according to claim 38, wherein said α2β1 integrin binding reagent is a peptide.

45. The kit according to claim 43, wherein said peptide comprises an amino acid sequence selected from the group consisting of SEQ ID NO:s 6 to 13.
